(19) United States
(12) Patent Application Publication
(10) Pub. No.: US 2026/0269084 A1
(43) Pub. Date: Sep. 10, 2026

(54) COMPUTATIONAL DRUG TARGET SELECTION

(71) Applicant: Recursion Pharmaceuticals, Inc., Salt Lake City, UT (US)

(72) Inventors: Daniel James CROWTHER, Dundee (GB); David NARGANES-CARLÓN, Dundee (GB)

(21) Appl. No.: 19/167,738

(22) PCT Filed: Mar. 22, 2024
(86) PCT No.: PCT/EP2024/057830

§ 371 Date: Sep. 22, 2025

(30) Foreign Application Priority Data
Mar. 23, 2023 (GB) 2304213.8

(51) Int. Cl. G16H 70/40; G16H 50/20

(52) U.S. Cl. CPC G16H 70/40; G16H 50/20

(57) ABSTRACT

The invention relates to computational drug target selection. The invention involves searching ingested biomedical publication data to identify occurrences of a term indicating defined genes and occurrences of a term indicating a defined disease. The invention involves defining a vocabulary including terms from the ingested biomedical publication data, including terms indicating the genes and the disease, and training a defined language model using the defined vocabulary to obtain a vector representation of each of the terms. The invention involves determining a likelihood score for each respective gene based on the vector representations, the likelihood score for the respective gene being a likelihood of the gene co-occurring with the disease in the biomedical publication data. The invention involves ranking each of the genes according to the respective likelihood scores, and selecting at least one of the genes as a drug target for the disease based on the ranking.

### FIELD OF THE INVENTION

The invention relates to methods and systems for the computational selection of target molecules or genes, e.g. drug targets, with which compounds or molecules, e.g. drugs, are to be designed to interact in an optimal manner.

### BACKGROUND

Drug discovery is the process of identifying candidate compounds for progression to the next stage of drug development, e.g. pre-clinical trials. Such candidate compounds are required to satisfy certain criteria for further development. Modern drug discovery involves the identification and optimisation of initial screening ‘hit’ compounds. In particular, such compounds need to be optimised relative to required criteria, which can include the optimisation of a number of different properties. The properties to be optimised can include, for instance: activity against a desired biological target; selectivity against non-desired biological targets; low probability of toxicity; and, good drug metabolism and pharmacokinetic properties (ADME). Only compounds satisfying the specified requirements become candidate compounds that can continue to the drug development process.

The identification, prioritisation and selection of biological or drug targets against which hit compounds are then to be optimised is therefore a critical step in the drug discovery process; indeed, target identification and prioritisation are the first key steps in the drug discovery process and in the development of new pharmaceutical agents. A drug target is something—typically a protein or nucleic acid, for instance—that exists in a living organism to which a drug interacts, e.g. binds. Such interaction with a drug causes a change in behaviour of the drug target. A promising drug target may be one that has an association with a particular disease under consideration, e.g. the drug target modifies the disease or plays a role in the pathophysiology of the disease.

The process of selecting a drug target is complicated by the vast number of potential drug targets that are available. For instance, for a human disease there are tens of thousands of genes expressing proteins that could conceivably be the targets for a new drug. Furthermore, as there are many thousands of human diseases classified by medicine then there are many millions—in particular, hundreds of millions—of possible target-disease combinations. The search space of solutions is therefore so large that it is unfeasible to experimentally test each combination or hypothesis.

Conventionally, drug targets have been identified on a case-by-case basis by medicinal chemists interpreting the published scientific literature, e.g. academic journals, and public databases. That is, traditionally a significant amount of target identification has been carried out by individual scientists using their expertise to interpret the scientific literature. However, a growing issue with this approach is in the shear wealth of public data, such as academic papers, that is available to be searched. There are tens of millions of published scientific papers in the area of life sciences, hundreds of thousands of genomes, and many hundreds of databases. Indeed, thousands of peer-reviewed articles are published every day without taking into account other sources of data, such as pre-prints and clinical trial reports. Clearly, it is therefore not possible for humans to keep abreast of all of the available sources of data when selecting drug targets. In other words, the increasing publication rates make it difficult to maintain an overview in order to identify promising new or existing drug targets.

Optimising the identification and selection of a drug target is crucial in optimising the overall drug discovery process. In particular, an optimal selection of drug target for a particular drug discovery project can increase the probability of identifying a candidate compound in less time, i.e. in fewer design cycles of the project. In turn, this reduces the associated time and/or cost associated with the particular project.

It is against this background to which the present invention is set.

### SUMMARY OF THE INVENTION

The present invention provides an improved method of identifying biological targets for drugs in association with particular diseases, in order to reduce the overall time and/or cost associated with the drug discovery process, e.g. to increase the efficiency of identifying a candidate compound as part of a particular drug discovery project. In addition, the invention provides methods for drug discovery. In particular, in methods comprising selecting at least one drug target, the methods may comprise undertaking a drug discovery project based on said at least one drug target; and optionally selecting and/or synthesising and/or testing potential therapeutic compounds against the at least one selected drug target.

According to an aspect of the invention there is provided a method for drug target selection. The method comprises at least some computer-implemented steps. The method comprises ingesting biomedical publication data from at least one biomedical publication data source that includes text-based biomedical publication documents. The method comprises searching the ingested biomedical publication data to identify each occurrence of a term indicating each of a defined plurality of genes. The method comprises searching the ingested biomedical publication data to identify each occurrence of a term indicating each of at least one defined disease. The method comprises defining a vocabulary including a plurality of terms from the ingested biomedical publication data, the terms including terms indicating each of the identified genes and each of the at least one identified disease. The method comprises training a defined language model using the defined vocabulary to obtain a vector representation of each of the plurality of terms. The method comprises, for each of the at least one identified disease: determining a likelihood score for each of the identified genes based on the obtained vector representations, the likelihood score for each identified gene being indicative of a likelihood of the respective identified gene co-occurring with the respective identified disease in the biomedical publication data; ranking each of the identified genes according to the respective determined likelihood scores; and, selecting at least one of the identified genes as a drug target for the respective identified disease based on the ranking.

The biomedical publication data relating to the biomedical publication documents may include one or more of: a title of the publication document; an abstract of the publication document; and, one or more keywords associated with the publication document.

The method may comprise defining, for each defined gene, one or more terms as referring to said defined gene. Searching the biomedical publication data may comprise searching the biomedical publication data for each occurrence of the one or more defined terms for each defined gene.

The method may comprise, for each identified gene, converting each of the identified occurrences of a defined term indicating the respective identified gene to a respective standard term. The terms indicating the respective identified gene in the defined vocabulary may be a single term in the form of the respective standard term.

The method may comprise defining, for each defined disease, one or more terms as referring to said defined disease. Searching the biomedical publication data may comprise searching the biomedical publication data for each occurrence of the one or more defined terms for each defined disease.

The method may comprise, for each identified disease, converting each of the identified occurrences of a defined term indicating the respective identified disease to a respective standard term. The terms indicating the respective identified disease in the defined vocabulary may be a single term in the form of the respective standard term.

Searching the ingested biomedical publication data may comprise identifying terms occurring at least a prescribed number of times in the ingested biomedical publication data. The plurality of terms from the ingested biomedical publication data included in the defined vocabulary may include the terms occurring at least the prescribed number of times. Optionally, the prescribed number may be 5, 6, 7, 8, 9, 10, 15, 20, 50 or 100.

The identified terms occurring at least the prescribed number of times may be unigrams. The method may comprise generating phrases of n-grams, where n is greater than or equal to two, based on the identified unigrams. The generated phrases may be included in the plurality of terms of the defined vocabulary.

Each generated phrase may be included in the defined vocabulary only if the respective phrase occurs at least the prescribed number of times in the ingested biomedical publication data.

Each generated phrase may be included in the defined vocabulary only if the respective phrase has a mutual information score greater than a prescribed threshold value.

Optionally, the prescribed threshold value may be between 0.5 and 0.9. Further optionally, the prescribed threshold value may be between 0.6 and 0.8. Further optionally, the prescribed threshold value may be 0.7.

The defined language model may be a neural network model.

A dimension of each vector representation may be equal to a number of neurons of a hidden layer of the neural network model. Optionally, the dimension may be 128, 256 or 512.

Values in the vector representation of a term from the biomedical publication data may be obtained from weights of the neurons of the trained neural network model.

The defined language model may take as input a context of each of the plurality of terms from the ingested biomedical publication data and may provide as output a prediction of the term corresponding to the context. Alternatively, the defined language model may take as input the plurality of terms from the ingested biomedical publication data and may provide as output a plurality of probability distributions of the context corresponding to the respective term.

The context of each term may include one or more words in the vicinity of each occurrence of the respective term in the biomedical publication data.

The vicinity may be a window of a prescribed size that defines the number of words either side of the occurrence of the term in the biomedical publication data that is to be included in the context. Optionally, the prescribed window size may be 1, 2, 3, 4, 5, 6, 7, 8, 9, 10, 15, 20 or 25 words.

The prescribed window size may be equal to a median number of words between occurrences of defined genes and occurrences of the at least one disease in the biomedical publication data. Optionally, the median number of words may be between 5 and 15. Further optionally, the median number may be 10.

The defined language model may use a Word2Vec algorithm. Optionally, the Word2Vec algorithm uses a contextual bag of words (CBOW) approach or a skip-gram approach.

For each identified disease, determining the likelihood score for each identified gene may comprise determining a distance metric between the vector representation of the respective disease and the vector representation of the respective gene.

The distance metric may be a cosine similarity metric.

Selecting at least one of the identified genes may comprise identifying a prescribed number of the identified genes having the highest likelihood scores as a top ranked set of genes, and may comprise selecting at least one of the identified genes from the top ranked set as a drug target.

The prescribed number may comprise a prescribed percentage of the total number of identified genes. Optionally, the prescribed number may be 0.01%, 0.1%, 0.2%, 0.5%, 1%, 2%, 5% or 10% of the total number of identified genes.

The prescribed number may comprise 5, 10, 20, 50 or 100 genes.

Selecting at least one of the identified genes may comprise selecting the identified gene having the highest likelihood score as a drug target.

The method may comprise designing a drug discovery project for developing a therapeutic drug for the at least one disease, the drug discovery project being based on the at least one selected gene being the drug target for the project. The drug discovery project may include a defined drug discovery process for developing a therapeutic drug for the at least one disease. Designing the drug discovery project may involve retrieving the defined drug discovery process, e.g. as a computer-implemented step.

The method may comprise undertaking the drug discovery project using the at least one selected gene as the drug target.

Undertaking the drug discovery project may comprise selecting and testing compounds against the at least one selected gene for therapeutic effects against the at least one disease.

Testing the compounds may comprise synthesising the respective compound and determining one or more properties of the compound. Optionally, the properties may be physical, chemical or biological properties of the respective compound.

The testing may be in vitro and/or in vivo testing.

The selection of compounds may comprise executing a trained machine learning model that predicts biological properties of compounds as a function of their structural features or fragments. The model may be trained using compounds for which the biological properties are known, e.g. because they have previously been synthesised and tested. The model may for instance use Bayesian optimisation. The model may be a neural network model. The model may take as input a fingerprint representation of a compound, which encodes the structural features of a compound in vector form, e.g. using an Extended Connectivity Fingerprint (ECFP). A selection of one or more compounds to synthesise and test may be performed based on the model predictions, e.g. the top scoring compounds according to a measure of their predicted biological properties relative to a defined desired property profile of a candidate compound (e.g. using a defined scoring function). The selected compounds may be synthesised and tested, and their biological properties determined during testing may be used to update/retrain the machine learning model as part of a feedback process ahead of a next iteration of executing the model to determine which compounds to select next for synthesis and testing.

The identified genes may include one or more human genes. Optionally, the identified genes may include one or more proteins encoded by said genes.

According to an aspect of the invention there is provided a method for identifying protein-protein interactions. The method comprises at least some computer-implemented steps, including the following. The method comprises ingesting biomedical publication data from at least one biomedical publication data source that includes text-based biomedical publication documents. The method comprises searching the ingested biomedical publication data to identify each occurrence of a term indicating each of a defined plurality of proteins. The method comprises defining a vocabulary including a plurality of terms from the ingested biomedical publication data, the terms including terms indicating each of the identified proteins. The method comprises training a defined language model using the defined vocabulary to obtain a vector representation of each of the plurality of terms. The method comprises, for each of the identified proteins: determining a likelihood score for each of the other identified proteins based on the obtained vector representations, the likelihood score for each of the other identified proteins being indicative of a likelihood of the respective other identified protein co-occurring with the respective identified protein in the biomedical publication data; ranking each of the identified other proteins according to the respective determined likelihood scores; and, identifying interactions between the other identified proteins and the identified protein based on the ranking. For instance, a high ranking score may indicate an identified interaction between two proteins.

According to another aspect of the invention there is provided a non-transitory, computer-readable storage medium storing instructions thereon that when executed by a computer processor causes the computer processor to perform the method defined above.

According to another aspect of the invention there is provided a computer device for drug target selection. The computer device comprises one or more computer processors configured to: ingest biomedical publication data from at least one biomedical publication data source that includes text-based biomedical publication documents; search the ingested biomedical publication data to identify each occurrence of a term indicating each of a defined plurality of genes, and search the ingested biomedical publication data to identify each occurrence of a term indicating each of at least one defined disease; define a vocabulary including a plurality of terms from the ingested biomedical publication data, the terms including terms indicating each of the identified genes and each of the at least one identified disease; train a defined language model using the defined vocabulary to obtain a vector representation of each of the plurality of terms; for each of the at least one identified disease: determine a likelihood score for each of the identified genes based on the obtained vector representations, the likelihood score for each identified gene being indicative of a likelihood of the respective identified gene co-occurring with the respective identified disease in the biomedical publication data; rank each of the identified genes according to the respective determined likelihood scores; and, select at least one of the identified genes as a drug target for the respective identified disease based on the ranking.

### BRIEF DESCRIPTION OF THE DRAWINGS

Examples of the invention will now be described with reference to the following drawings, in which:

FIG. 1 summarises the steps of a computational drug selection method in accordance with the invention;

FIG. 2 shows results of an experiment implementing the method of FIG. 1 to prioritise target-disease associations;

FIG. 3 illustrates results of a Uniform Manifold Approximation and Projection (UMAP) of word embeddings, obtained using the method FIG. 1, for the human protein-coding, published genome;

FIG. 4 shows results of an experiment implementing the method of FIG. 1 to prioritise protein-protein interactions; and,

FIG. 5 shows results of an experiment implementing the method of FIG. 1 to prioritise novel mechanisms of action of drugs.

### DETAILED DESCRIPTION

Molecular or drug design can be considered a multi-dimensional optimisation problem that uses the hypothesis generation and experimentation cycle to advance knowledge. Each compound design can be considered a hypothesis which is falsified in experimentation. The experimental results are represented as structure-activity relationships, which construct a landscape of hypotheses as to which chemical structure is likely to contain the desired characteristics. The process of drug design is also an optimisation problem as each project needs a defined product profile—i.e. drug target function—of desired, specified attributes against which hit compounds are analysed.

The drug discovery process is often performed in iterations known as design cycles. At each iteration, a set of molecules or compounds is synthesised, and their biological properties are measured. The activities are analysed, and a new set of compounds is proposed, based on what has been learned from previous iterations. This process is repeated until a clinical candidate is found. As well as activity, the measured biological properties can include one or more of selectivity, toxicity, absorption, distribution, metabolism, and excretion.

The drug discovery process is generally time consuming and expensive. Efficiencies that can be found at any stage of the process can therefore help in reducing the time and cost associated with a drug discovery project. Pharmaceutical companies are actively looking for ways to reduce their attrition rates, the time taken for drug development, and the associated development costs.

The selection of drug targets for developing a new drug is the first decision—and arguably the most important single decision—in the drug discovery process. Historically, target identification has been broadly carried out on a case-by-case basis, based on the scientific interpretation of the available literature. However, thousands of peer-reviewed articles are published every day in addition to the publication of pre-prints, patent document data, and clinical trial reports. The online resource PubMed® allows search of, and access to, publication documents in the field of life sciences and biomedical information. PubMed® alone contains access to tens of millions of publication documents—in particular, more than 34 million publications as of 2022—and the scientific output doubles every nine years or so. This creates a corpus of ‘undiscovered public knowledge’ as it is clearly not feasible for a human, e.g. a medicinal chemist, to keep pace with all of the developments in the published literature. In turn, this makes it more difficult for a human to make an informed decision on the identification and selection of drug targets based on the available literature. Indeed, historically only around 10% of drug targets progress through clinical trials, and this success rate may be even lower for novel drug targets.

The vast search space of possible drug targets also makes it difficult for a human to make optimal selections. For a human disease, with tens of thousands of genes that could in theory play a role in the behaviour of a particular disease, there are many millions of gene-disease combinations that could be investigated but which in practice is clearly not feasible.

The above issues mean that the use of computational methods to analyse the vast amount of available information and the huge number of possible gene-disease combinations becomes an attractive proposition. In particular, there is a high demand for machine learning (ML), artificial intelligence (AI), and other computational methods to exploit the current knowledge and facilitate the maintenance of an overview of this overwhelming volume of literature with a view to optimising identification and selection of drug targets. Indeed, several approaches have shown that ML can speed up drug discovery at multiple stages.

A limiting factor for the prioritisation of gene-disease (or target-disease) hypothecation using computational methods is that most of the information available in the published literature is not in the form of machine-interpretable data. Most biomedical knowledge is published as text, which is difficult to analyse using traditional statistical approaches. Publications contain valuable knowledge regarding inferences interpreted by the scientific community.

The primary source of machine-interpretable data for computational approaches is currently structured property databases; however, such databases only encompass a tiny fraction of the knowledge present in the published literature. Clearly, in order to provide as accurate and useful insights as possible, computational methods need to be provided with as much of the available information as possible.

The present invention advantageously provides an approach for encoding biomedical knowledge from the published literature (that is published as text) as word embeddings without human labelling or supervision. The present invention also advantageously provides an approach for capturing different drug discovery concepts such as clinical tractability, disease association and biochemical pathways, in particular via the use of language models. Specifically, the approach of the present invention beneficially prioritises hypotheses, e.g. relating to gene-disease associations, prior to them being first reported in the published literature. This means that therapeutic drug targets associated with certain diseases can be identified, prioritised and selected for clinical trials several years earlier than would otherwise be the case, thus potentially leading to a significant reduction in the cost and time taken to develop a new drug for a particular disease. The invention provides for the prioritisation of under-explored targets and a scalable system that accelerates early-stage target ranking.

The invention involves training unsupervised language models on the published biomedical literature. These models are used to prioritise or suggest novel drug discovery scientific hypotheses, i.e. ones that have not yet been published. In particular, the language models perform drug discovery tasks such as gene-disease prioritisation and target identification/selection, but also protein-protein interaction prediction. Drug targets with genetic evidence, e.g. undiscovered from the literature, are twice as likely to succeed in clinical trials. The invention prioritises gene-disease associations and selection of drug targets for specific diseases before they are stated in the literature for the first time. In this way, the invention allows for a determination of what ought to, or should, be described in the published literature by way of gene-disease associations and novel drug targets, based on the published literature as a whole, but is not. This allows for drug-like molecules or compounds to be conceived, synthesised and tested against such targets earlier than they otherwise would be, thereby resulting in significant time and cost savings to develop a new drug for a particular disease.

The invention therefore may be regarded as beneficially providing an improved process for drug design/development that is achieved via the identification and selection of better drug targets that allow candidate compounds that have therapeutic effects against particular diseases to be identified more efficiently. A drug discovery process may be undertaken that seeks to identify (hit/lead/candidate) compounds that have a therapeutic effect against a drug target identified via the better selection process for a defined disease. The process to identify such compounds may include one or more standard processes, e.g. virtual screening or high throughput screening, or one or more non-standard process, e.g. active learning techniques using a machine learning model to predict biological properties of compounds as a function of structural features or fragments of compounds (possibly using a feedback loop to update the model based on biological properties obtained via testing of selected compounds). However, the benefit of identifying candidate compounds in a more time and cost efficient manner is achieved irrespective of the particular process in this regard, and instead achieved by virtue of the fact that the drug discovery process is performed using a drug target identified according to the identification and selection process described herein.

The invention in particular involves encoding the published literature as word embeddings or vector representations of words. Without any direct insertion of a priori biomedical knowledge, these embeddings capture complex biomedical interactions, such as drug target classes, tractability, involvement in disease phenotypes, and protein-protein interactions. As discussed below, implementation of the trained language models of the invention during experimentation shows that the language models ranked novel hypotheses several years before their publication in top-tier journals. This prioritisation indicates that knowledge regarding ‘future’ discoveries is to some extent embedded in past/current publications.

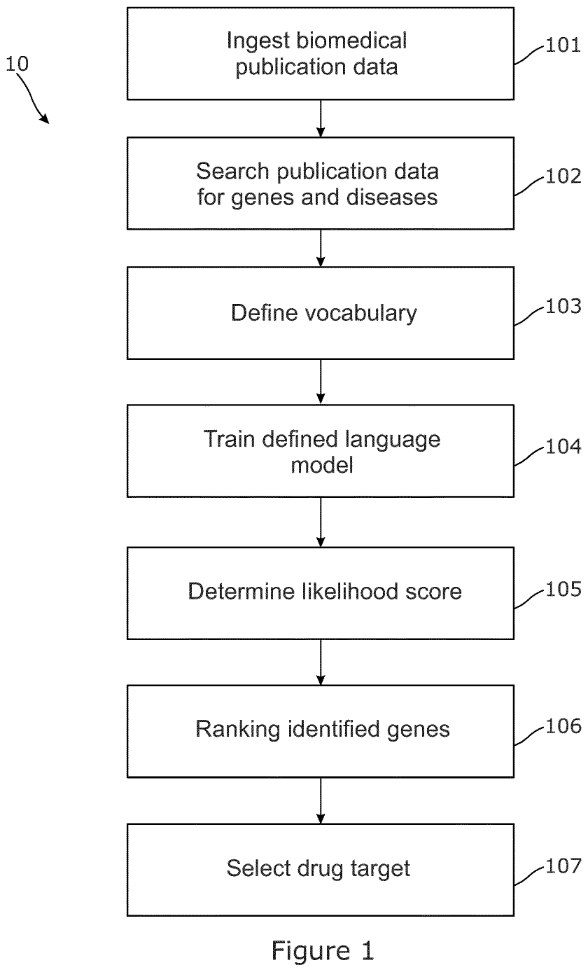

FIG. 1 summarises the steps of a computational drug target selection method 10 according to the invention. In accordance with the present invention, a first step 101 of the computational drug target selection method comprises ingesting, receiving or downloading biomedical publication data from at least one biomedical publication data source. For instance, the biomedical publication data source may be an online publication data source, and may include a database such as PubMed® that has access to millions of publication documents in the form of academic or journal articles in the particular field of interest. Biomedical publication data may additionally or alternatively be ingested from other sources such as published clinical trial report data, published patent document data and/or published pre-prints of articles. It will be understood that biomedical publication data may be obtained and ingested from any suitable source of such biomedical publication data, and from any number of these suitable sources.

The biomedical publication data is in particular text-based. That is, the biomedical publication data includes biomedical information in the form of words, phrases, sentences, etc., (rather than in the form of a structured database, for instance). The information is therefore in the form of alphanumeric character expressions, i.e. including letters (possibly from various alphabets), numbers, other symbols, etc.

The information included in the ingested publication data for a given publication document may depend on the particular type of document or the particular source from which the publication data is obtained. For instance, the publication data may be restricted to the data available as open source data for a given publication document, with further information only being accessible behind a paywall.

In this regard, biomedical publication data in the form of journal articles, for instance, may be limited to the information that is not behind a paywall, typically including a title, abstract and keywords of an article, as well as details of the journal, publication date, etc.

The ingested publication data relates to a plurality of different publication documents, i.e. each publication document has publication data associated with it. The publication data associated with at least some of the plurality of publication documents may typically include a publication date of, or associated with, those publication documents. The publication date could be a specific day, month or year of publication of the relevant document, for instance. The publication data of a particular publication document may include authors, journal name, etc.

A next step 102 of the computational drug target selection method 10 includes searching the biomedical publication data to identify terms of interest, where terms can be regarded as character expressions or sequences corresponding to words, phrases, sentences, symbols corresponding to biomedical entities or concepts, etc. In particular, the present method is concerned with identifying any mention of any type of gene, or any gene of interest. More generally, the present method is concerned with identifying mentions of entities that may be the target for a particular drug aimed at providing therapy for a particular disease. In the described example, said drug targets are genes. The present method is also concerned with identifying any mention of particular diseases of interest.

In one example, the names of one or more drug targets of interest, e.g. genes of interest, are defined (e.g. by a user) and the content of the biomedical publication data is automatically searched for the defined gene names. For instance, if the defined name of one of the genes is found in the publication data associated with a particular publication document, then that gene may be regarded as being associated with, or linked to, the particular publication document. The defined name for a drug target may for example be an approved symbol, e.g. an approved gene symbol, according to an accepted nomenclature. Here, ‘gene symbol’ is used to refer to the approved symbol for a particular gene from any of the 19084 human, protein-coding genes accepted by the HUGO Gene Nomenclature Committee; however, it will be understood that this is purely for illustrative purposes and is non-limiting.

A significant obstacle for the automatic analysis of the biomedical literature by computational methods is in the use of non-redundant alternative gene (drug target) synonyms, symbols, and acronyms from different competing sources that can have other meanings in other areas of research. That is, in the literature a single drug target, such as a gene, can be referred to in a number of different ways that are accepted in the field. It can also be the case that one or more of the synonyms for a particular drug target coincides with, or is part of, the term or expression for an entirely different concept, or have an entirely different meaning, in a different context. These factors make it difficult to unambiguously determine via automatic (computational) analysis which publication documents that include references coinciding with a name for a gene do in fact refer to that gene.

In order to identify references in the publication data to drug targets of interest that are referred to in a number of different ways, by a number of different names, or in different languages, the method may include defining, for each drug target, one or more terms, character expressions or synonyms as referring to that drug target or gene. These character expressions may be defined by a user, and may include any suitable characters that can be searched computationally. For instance, suitable characters may include letters used in one or more natural languages, or other types of symbols. Then, for each gene, searching the publication data includes searching the biomedical publication data to identify each occurrence of the one or more defined terms, character expressions or synonyms for each gene of interest.

In this context, a ‘gene synonym’ may refer to any of the possible gene name variations by which the scientific community refers to, or has referred to, a given gene. Approved gene symbols—as defined above—are also included in the gene synonyms. As an illustrative example, ‘EGFR’ is the approved gene symbol whereas ‘EGFR’, ‘Epidermal Growth Factor Receptor’, ‘ERBB1’, ‘ErbB-1’, ‘c-erbB1’, ‘HER1’, and ‘ERBB’ may be the gene synonyms.

A similar approach may be taken for each disease of interest. That is, each disease of interest may have one or more defined terms, character expressions or synonyms associated therewith, e.g. as defined by a user. As a non-limiting illustrative example, disease names and their synonyms may be obtained from the Medical Subject Headings (MeSH) ontology at the Bioportal. MeSH ontology contains 4819 different disease nodes at different levels of the ontology. For instance, a dictionary for each disease may be created with the preferred and alternative names. For each disease of interest, searching the biomedical publication data includes searching the biomedical publication data to identify each occurrence of the one or more defined terms, character expressions or synonyms for each disease of interest.

The different character expressions defined as potentially referring to a particular gene or disease may be obtained from different sources. For instance, in a case in which human genes are of interest, the various character expressions, i.e. synonyms, for different human genes may be gathered from different sources to sample the potential publication documents mentioning human gene names.

A next step 103 of the drug target selection method 10 comprises defining a vocabulary including a plurality of terms from the ingested biomedical publication data. The vocabulary is text-based, like the information in the ingested data. The vocabulary is to be used to encode certain text-based terms from the biomedical publication data as a vector representation that includes contextual information for the terms obtained from the biomedical publication data. This may be referred to as a word embedding or term embedding, as described in greater detail below.

The terms included in the vocabulary include terms indicating each of the genes of interest. For instance, the terms may include the approved symbol for each gene of interest. As such, the method may involve converting each identified occurrence of a term indicating a gene in the publication data to the approved gene symbol (or other default or standard synonym) for inclusion in the vocabulary. The terms in the vocabulary also include terms indicating each of the defined diseases of interest, e.g. a default or standard identifier or synonym for each disease. Again, each identified occurrence of a term indicating a disease of interest in the ingested data may be converted to the standard identifier for said disease for inclusion in the vocabulary.

In order that each of the genes and diseases identified in the biomedical publication data can be encoded into an appropriate vector representation, not only is each occurrence/mention of the genes and diseases in the publication data needed in the vocabulary, but the context in which each of these terms appears in the publication data needs to be captured in the vocabulary. The vocabulary may therefore include a certain number of words or terms either side of an occurrence/mention of a gene or disease in the publication data, i.e. surrounding words or other words in the vicinity. This number of words could be a defined number, e.g. 5, 10, 15, etc., or it could be all of the other words in a sentence where the gene or disease is mentioned. The positioning of other words/terms relative to gene or disease term, and/or relative to one another, may be captured.

The vocabulary may further include other words/terms (i.e. other than those referring to the genes or diseases) that appear/occur a certain number of times in the ingested publication data. The method may therefore involve searching the ingested biomedical publication data to identify all words/terms that appear at least a defined/prescribed number of times, e.g. at least 5, 6, 7, 8, 9, 10, 15, 20, 50 or 100 times, or any other suitable number (which may depend on the size of the ingested data being searched). The vocabulary therefore includes all words/terms that occur at least a defined number of times, as well as the normalised/standardised identifiers for each gene and disease of interest (irrespective or independent of the number of times each of these occur). The vocabulary may further include the context in which each of these words/terms that appear at least the defined number of times in the publication data, in a similar manner to the context of the genes and diseases.

If the searching step of the method identifies individual words, then an additional, optional step may involve further processing the searching results to obtain phrases, of more than one word/expression, e.g., phrases that appear at least a/the prescribed number of times. In particular, searching the publication data may provide individual words or unigrams appearing a certain number of times, and further processing to obtain n-grams, where n>1, that appear the certain number of times may be performed. In addition to appearing at least a prescribed number of times in the text corpus (ingested publication data), the phrases being generated may also have at least a minimum level of mutual information, and may have a certain phrase depth. The minimum level of mutual information can be any suitable value. For instance, a normalised mutual information score may be greater than a prescribed threshold value, e.g. between 0.5 and 0.9, between 0.6 and 0.8, approximately 0.7, or any other suitable value.

In an illustrative example, if the phrase ‘g protein coupled receptor’ appears at least the prescribed number of time in the publication data and has at least the minimum mutual information, then the phrasing process may be applied once to obtain ‘g_protein coupled_receptor’ (i.e. two bi-grams rather than four unigrams), and then applied a second time to obtain ‘g_protein_coupled_receptor’. This single term/token may then be included in the vocabulary instead of the unigrams (individual words/expressions).

A next step 104 of the method 100 involves training a defined language model using the defined vocabulary to obtain a vector representation (or distributed representation) of each of the plurality of terms. As mentioned above, this is referred to as word embedding or term embedding. As is known, word embedding provides a method for representing a defined vocabulary, and captures context of an instance of a word/term in text data, relationships to other words/terms, etc.

The defined language model to be used to learn the word embeddings/vector representation of the words/terms in the defined vocabulary may in particular use a neural network comprising input, hidden and output layers. The neural network may be a shallow neural network. In particular, in the described example the known Word2Vec algorithm/technique is used to learn the embeddings of the words/terms in the defined vocabulary using a shallow neural network.

Training the defined language model involves learning values of hidden layer weights using the words/terms and their context in the defined vocabulary, i.e. the training set for the model is the words/terms and their context in the defined vocabulary. In one approach—namely, a contextual bag of words (CBOW) approach in Word2Vec—the context of a word/term is the input to the neural network and the word/term is the output. In another approach—namely, a skip gram (SG) approach in Word2Vec—a word/term is the input to the neural network and the context of the word/term is the output. The neural network may be trained to determine values of the hidden layer weights based on this training set (i.e. input/output pairs of data) in any known manner, e.g. back propagation. A neural network that includes trained weights for each word/term in the defined vocabulary is obtained.

Once the language model has been trained, it can therefore be used to either: predict a word/term corresponding to context (surrounding words) provided as input; or, predict the context (surrounding words) corresponding to a word/term provided as input.

The vector representation of the words/terms in the defined vocabulary may take any suitable form. In particular, the vectors may be of any suitable dimension/embedding size, e.g. 128, 256, 512, or any other suitable size. The dimension of the vector representation may correspond to the dimension (number of neurons) of the hidden layer of the neural network. The values of the vector for a particular word/term may be obtained from (be based on) the trained weight values of the neural network.

A next step 105 of the method 10 involves determining likelihood scores between respective genes and diseases. In particular, this step provides a measure or metric indicating how likely it is that a particular gene and disease are associated with one another even though they have never previously been mentioned together (co-occurred) in the published literature (the biomedical publication data). Specifically, for each disease of interest, a likelihood score is determined for each gene of interest based on the obtained vector representations. That is, for a particular disease and a particular gene, a likelihood score is determined using the respective vector representations of said particular disease and particular gene, the likelihood score being indicative of how likely it is that said particular disease and particular gene co-occur in the publication data.

The likelihood score can be regarded as a measure of how similar the vector representations of two biomedical entities (e.g. gene and disease) are, which in turn can be regarded as a measure of how close the two vectors are in vector space (i.e. a vector distance metric). The distance metric used may for instance be a cosine similarity metric, where the cosine similarity cos 0 is defined according to:

$$cos θ = \frac{A \cdot B}{‖ A ‖ ‖ B ‖}$$

where A, B are the vector representations of the particular disease and gene under consideration. Note that the cosine similarity metric can take a value between 0 and 1, and the more similar two vector representations are (i.e. the more likely a disease and gene are to co-occur) the higher the cosine similarity value (associated with the gene) is.

Although a particular gene and a particular disease may never have previously been mentioned together, i.e. they have not co-occurred in the published literature, the particular gene may have a relatively high likelihood score in connection with the particular disease if the particular gene and disease have appeared in the same context in the published literature. For instance, if a particular word/term is mentioned in association with the particular gene in the published literature, and the particular word/term is also mentioned in association with the particular disease in the published literature, then it may be determined that there is an association between the particular gene and disease.

A next step 106 of the method 10 involves, for each disease of interest, ranking each of the identified genes of interest according to their respective determined likelihood scores. In particular, the genes can be ranked in order of highest to lowest likelihood score, e.g. highest to lowest cosine similarity value, associated therewith.

A next step 107 of the method 10 involves, for each disease of interest, selecting at least one of the identified genes as a drug target for the respective identified disease based on the ranking. The selection may in particular be an automatic selection. In one example, one or more of the highest-ranking genes, i.e. those with the highest likelihood scores, may be selected, e.g. the top 1, 2, 3, 4, 5, etc. scoring genes.

In another example, further analysis or evaluation may be performed prior to selection. For instance, a prescribed number of the highest scoring genes may be identified (a ‘top ranked set’), and further evaluation of said identified genes may be performed to make the selection. The prescribed number could be for instance a prescribed percentage of the total number of identified genes, e.g. 0.01%, 0.1%, 0.2%, 0.5%, 1%, 2%, 5% 10%, etc. of the total number of identified genes. The prescribed number could alternatively be any suitable number, e.g. 5, 10, 20, 50 100, etc. genes. The further evaluation may take into account other factors as to why a particular identified gene may or may not make a suitable drug target. In particular, while a particular identified gene may have a high likelihood score, there may be other reasons why said gene would not be an appropriate drug target, and so should not be selected as such. The further evaluation may be performed automatically, e.g. to analyse the identified genes in the top ranked set against one or more defined further criteria.

A further step of the of the method in accordance with the invention may involve, for one of the identified diseases, designing a drug discovery project for developing a therapeutic drug for said identified disease. In particular, the drug discovery project is based on the at least one selected gene being the drug target for the project. The drug discovery project may involve selecting and testing compounds against the selected gene(s) to determine one or more properties of the compounds in connection with the gene(s). These could be physical, chemical or biological properties of the respective compounds. The method of the invention can incorporate undertaking the designed drug discovery project. Testing the selected compounds may involve in vitro and/or in vivo testing. For instance, testing may involve synthesising the selected compounds, e.g. in a laboratory, and performing various measurements, etc. to determine their properties, e.g. binding activity against the identified gene. The results associated with one or more tested compounds may be used to inform the selection of one or more further compounds to test against the drug target (identified gene), with the aim being to identify a compound having a desired/optimal property profile against the drug target, so that it may be used as a therapeutic drug in association with the disease of interest.

The steps of the method of the invention that are to be performed computationally (e.g. steps 101 to 107) may be implemented on any suitable computing device, for instance by one or more functional units or modules implemented on one or more computer processors. Such functional units may be provided by suitable software running on any suitable computing substrate using conventional or customer processors and memory. The one or more functional units may use a common computing substrate (for example, they may run on the same server) or separate substrates, or one or both may themselves be distributed between multiple computing devices. A computer memory may store instructions for performing the method, and the processor(s) may execute the stored instructions to perform the method.

Many modifications may be made to the above-described examples without departing from the scope of the appended claims.

Although the examples above describe the invention in relation to gene-disease association/prioritisation, it will be understood that a corresponding approach can be taken to identify associations between different proteins in the published literature, to identify associations between proteins and drug-like compounds that modulate their activity, etc.

In a specific example implementation of the computational drug target selection method 10 of the present invention, the biomedical publication or input data may consist of approximately 19.5 million abstracts from PubMed that mention any synonyms of any human genes or any human diseases in their title or abstract. Abstracts tagged in PubMed as ‘Commentary’, ‘Correction’, and ‘Corrigendum’ may be discarded. Genes and diseases may be normalised to standardised identifiers, where normalising means canonicalising all synonyms of a concept. For example, ‘Her2’, ‘ERBB2’, and ‘Neu’ can be normalised into their Ensembl identifier; ‘non-insulin-dependent diabetes’, ‘diabetes mellitus, type II’, ‘T2DM’ can be normalised to the Medical Subject Headings identifier. Synonyms may be normalised for several reasons. Normalising entities reduces the vocabulary size. Independent research groups use different gene synonyms in different research contexts (genetics with official gene symbols versus biochemistry articles), and the canonicalisation aggregates the information into a single token that can be used can be used to generate a genome-wide ranking of 20,000 human protein-coding genes rather than n_synonyms times number of genes. In this specific example implementation, the biomedical entity normalisation reduced the mentions of all gene synonyms 5-fold from 102,719 to 19,229 human protein-coding gene identifiers. The same occurred with the disease synonyms: it was reduced 10-fold, from 53,317 synonyms to 4,819 human disease identifiers. A correct pre-processing of the corpus (i.e. input or publication data) can substantially improve the performance of language models.

In this specific example, the vocabulary for the Word2Vec models consisted of all words that occurred more than 10 times, and normalised gene and disease identifiers, independently of the number of mentions. As a result of this threshold, the historical models (which each cover different year ranges) contain different vocabularies. If a gene is never mentioned in the training corpus (ingested biomedical publication data), then it will not be included in the vocabulary of the language model. For example, there were 13,676 distinct human genes mentioned in the training corpus until the year 2005 and 16,533 different human genes mentioned until the present day, which 86% of the total number of 19,229 human protein-coding genes.

The normalised gene and disease mentions refer to the mapping from any references in the text to a unique identifier from the HUGO or the Medical Subheadings Ontology (MeSH), respectively. Leading statements such as ‘Background:’, ‘Abstract:‘ and’Introduction:’ were also removed.

In the specific example implementation, phrases may be generated using a minimum phrase count of 10, normalised mutual information score higher than 0.7 and phrase depth up to 3 times. The phrasing process may be repeated three times, allowing the generation of up to 8 grams. For example, the phrase ‘g protein coupled receptor’ appeared >10 times in the training corpus, and its normalised mutual information score was >0.7. The phrasing turned ‘g protein coupled receptor’ into ‘g_protein coupled_receptor’ and then into ‘g_protein_coupled_receptor’.

The text may be lower-cased and deaccented. Floating decimals and percentage numbers may be converted with regular expressions into the word ‘<number>’ to reduce the vocabulary. Stop words may be kept as they only accounted for approximately 100 tokens in the half-million token vocabulary.

In this specific example, the Word2Vec algorithm from the Python library Gensim was used. Different combinations of algorithm, hyperparameters were used to see which combination captured better gene target to disease indication analogies. In particular, the different combination of hyperparameters tested are shown in Table 1. A window size of 10 was chosen, corresponding to the median distance (in words) between diseases and genes in PubMed. An embedding size of 255 minimised the Pairwise Inner Product loss according to ‘On the Dimensionality of Word Embedding’, Yin and Shen, Advances in Neural Information Processing Systems, vol. 31 (2018), although a grid search of 128, 256 and 512 was tried. The rest of the hyperparameters were defined left as default. The learning rate decreased linearly from 0.01 to 0.0001 in 40 epochs; a context or distance window of 5, 10 and 15 tokens; subsampling with a 10−4 threshold, which subsamples approximately the 400 most frequent tokens in the vocabulary; and both variants of Word2Vec: skip-gram (SG) and contextual bag of words (CBOW). The model that maximised a curated dataset of target to disease analogies was chosen, namely, CBOW with an embedding size of 256 and a window distance of 10 (see Table 1).

Table 1 shows a hyperparameter grid search for the Word2Vec different models. The different hyper-parameters used were the task of the Word2Vec model, either contextual bag-of-words (CBOW) or the skip-gram (SG); the size of the embedding vectors or the number of variables in the word vectors as multiples of 2 (128, 256 and 512); and the window distance, the number of tokens (words or phrases) between the pivot centre word and the surrounding words that are attempted to be predicted by the algorithm. Entries in the table represent the vocabulary ranking of the correct answer for analogies of the type: target A is to disease A as to target B is to disease B like in the original publication of ‘Efficient Estimation of Word Representations in Vector Space’, Mikolov et al., ArXiv13013781 Cs (2013).

TABLE 1

| | | Window Distance | | |
|---|---|---|---|---|
| Task | Embedding Size | 5 | 10 | 15 |
| CBOW | 128 | 1052 | 856 | 899 |
| CBOW | 256 | 987 | 771 | 813 |
| CBOW | 512 | 1123 | 903 | 964 |
| SG | 128 | 954 | 899 | 904 |
| SG | 256 | 943 | 792 | 891 |
| SG | 512 | 923 | 851 | 903 |

In the following, specific non-limiting examples using methods and systems in accordance with the invention are described, e.g. the specific example implementation described above.

### Example 1

In a first example, the method of the present invention was tested for its capability to perform target-disease prioritisation.

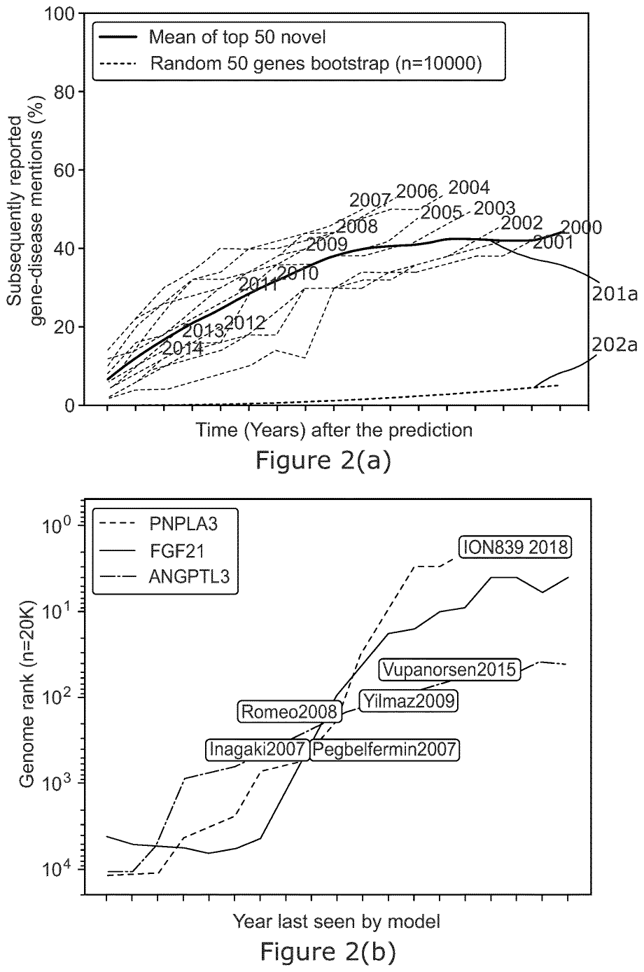

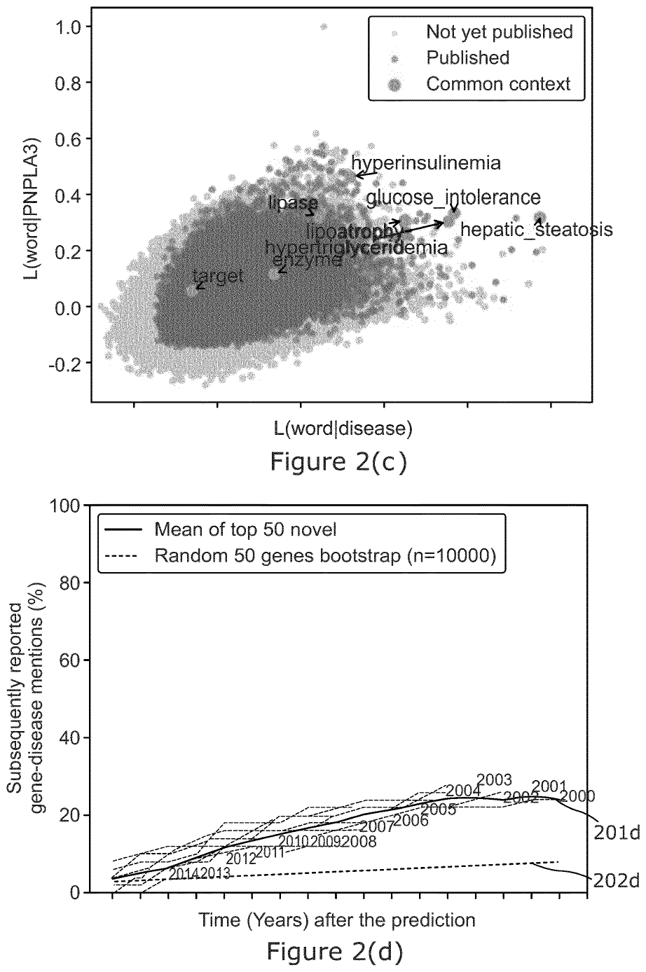

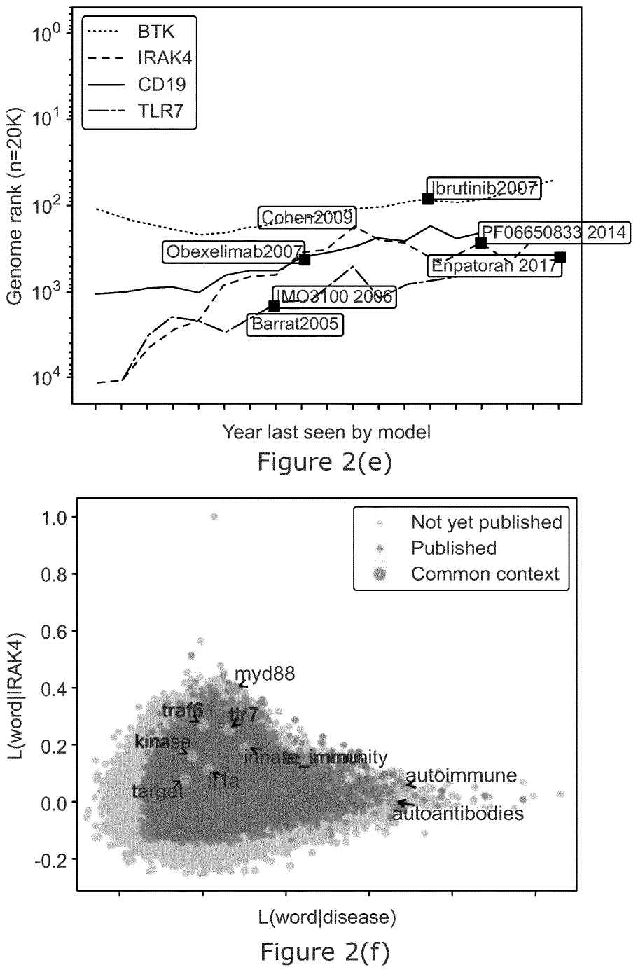

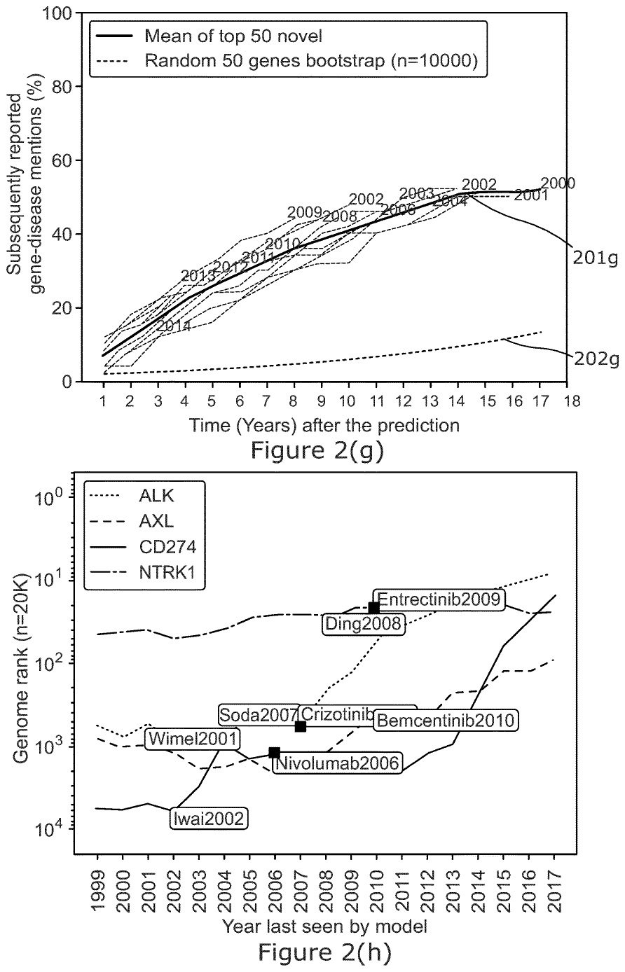

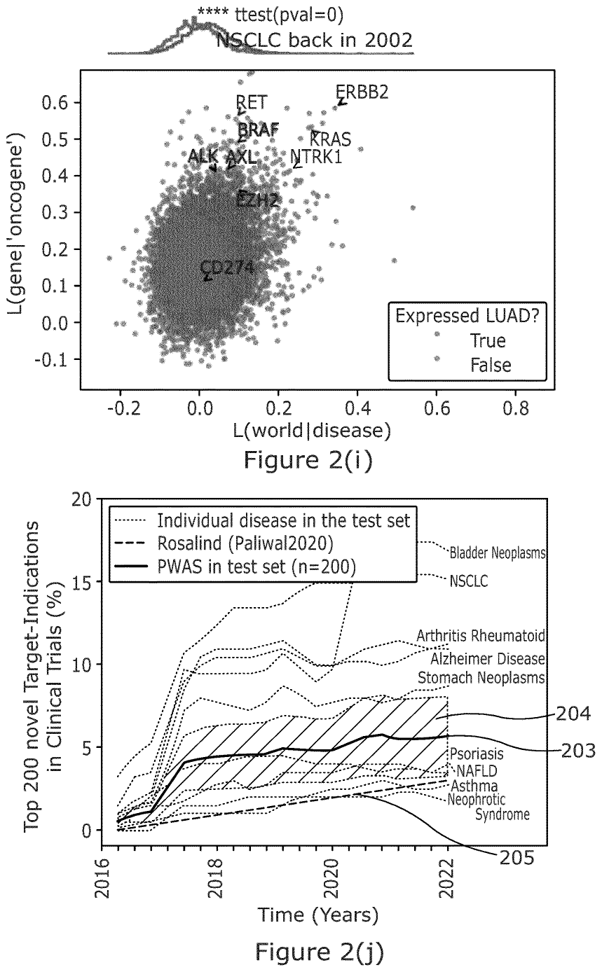

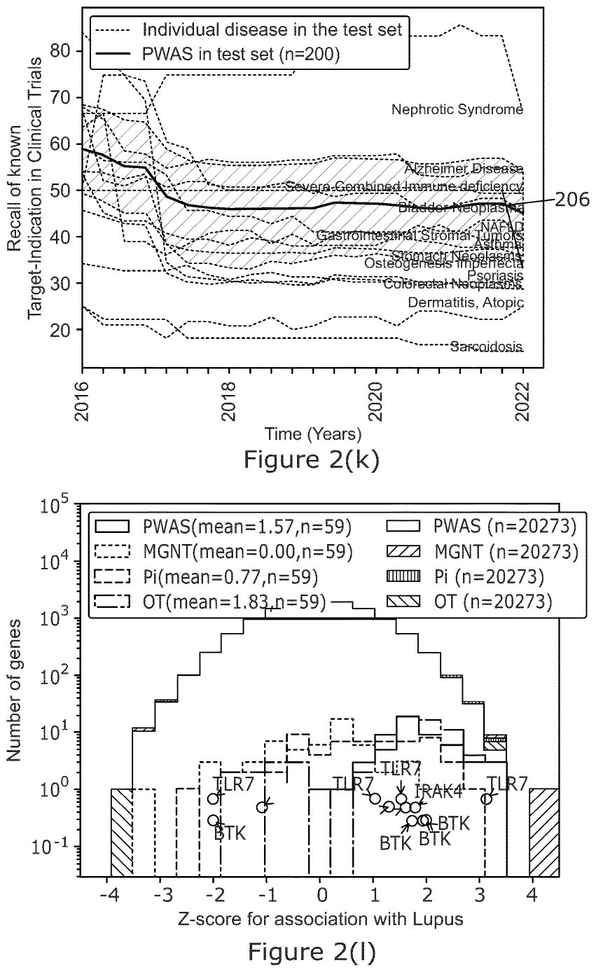

FIG. 2 shows results of testing/experimentation for the described method to prioritise target-disease associations/pairing not yet reported in the literature. FIGS. 2(a), 2(d) and 2(g) show predicted gene-disease associations on historical datasets. Each dotted line uses abstracts published before that year to make prospective predictions. Dotted lines plot the cumulative percentage of predicted genes subsequently reported in the years following their predictions; earlier predictions are analysed over longer testing periods. The results are averaged—indicated respectively by the plots 201a, 201d, 201g and compared to a random bootstrap sampling of 50 genes in 10,000 iterations, indicated by respectively by the plots 202a, 202d, 202g. FIG. 2(a) is for non-alcoholic fatty liver disease or NAFLD, FIG. 2(d) is for systemic lupus erythematosus (SLE), and FIG. 2(g) is for non-small cell lung carcinoma or NSCLC.

FIGS. 2(b), 2(e) and 2(h) show historical gene ranking scores. Each subplot shows genome-wide ranking scores (n=19,229) from 1999 until 2017. The name of the first author and the year of publication are in the grey-filled square next to the ranking plot. The name of the drug and the year of first reported evidence are in the white-filled square next to the ranking plot. FIG. 2(b) is for NAFLD, FIG. 2(e) is for SLE, and FIG. 2(h) is for NSCLC.

FIGS. 2(c), 2(f) and 2(i) are likelihood plots for interpretability. FIG. 2(i) shows the likelihood of some oncogene targets of NSCLC co-occurring with NSCLC (x-axis) and the word ‘oncogene’ (y-axis) becoming darker according to the mean levels of gene expression from The Cancer Genome Atlas (TCGA). The rankings are enriched (t-test with p-value=1e−189) for genes expressed in lung adenocarcinoma cells (threshold 0.1 transcripts per million, TPM).

FIG. 2(j) shows subsequent target-disease links entering trials. Lines labelled with a disease represent the cumulative percentage of top-scoring target-indication pairs subsequently reported in clinical trials following their predictions in 2016. Each line represents a unique disease in the test set (n=200). These results are averaged, as indicated by plot 203, with standard deviation indicated by the shaded region 204. The metrics for the 5-year window prediction from 2015 described by the Rosalind method from ‘Preclinical validation of therapeutic targets predicted by tensor factorization on heterogeneous graphs’, Paliwal et al., Sci. Rep. 10, 18250 (2020) are indicated by the plot 205. FIG. 2(k) shows prospective recall at 200. As more target-indication hypotheses are tested, the recall decreases over the subsequent years (x-axis) as more target-indication pairs enter clinical trials. The recall for 25 out of the 200 diseases in the test set is plotted with their names indicated. The mean for the 200 diseases is the plot 206. FIG. 2(l) shows target z-scoring for SLE. Four rankings are compared: the ranking according to the approach described herein (PWAS), MAGENTA (MGNT), Pi (Priority Index) and Open Targets (OT). Outer-shaded histograms show the normalised z-scores for 19,229 human protein-coding genes. Inner histograms show normalised z-scores for 60 SLE targets with FDA-approved drugs or in clinical trials.

The approach for obtaining the results in FIG. 2 is now described, along with discussion thereof. Multiple Word2Vec language models were trained on 19.5 million biomedical abstracts to obtain embeddings for a vocabulary of ~2,000,000 phrases. The abstracts were pre-processed to remove unnecessary characters and to normalise biomedical entities like genes and disease mentions. The entity normalisation reduced the gene synonym mentions from 102,719 to 19,229 identifiers for human protein-coding genes and 53,317 disease synonym mentions to 4,819 human disease identifiers. These language models were saved at the end of each year to create a retrospective analysis. Language models use co-occurrence information on a text corpus to estimate the likelihood of two words or phrases co-occurring despite never being mentioned together in the training set. The likelihood scores can be used to rank gene-disease hypotheses despite never having been explicitly stated before. Language models were trained on literature up to various points in the past, and, subsequently, their ranking was evaluated on prospective gene-disease associations published in the literature. Specifically, 28 different historical text corpora consisting of abstracts published between 1995 and 2022 were generated, each incrementing by one year in the cut-off date. Independent models were trained with those historical datasets, and they were used to prioritise associations between genes and diseases that were likely to be reported in future (test) years.

For each year, the language models were used to make a prediction. It was tested which percentage of the top-ranked genes without prior publications were subsequently reported in the literature (FIGS. 2(a), 2(d) and 2(g) for NAFLD, SLE and NSCLC, respectively). The dotted lines correspond to the predictions made by a different language model trained with one of the 28 different historical datasets. For example, the dotted line labelled with the year 2012 in FIG. 2(a) depicts the percentage of the top 50 novel predictions from the 2012 model that were found to be associated in the following years. The cumulative precisions are averaged (plots 201a, 201d, 201g in FIGS. 2(a), 2(d) and 2(g), respectively) and compared to random bootstrap sampling of 50 genes without replacement in 10,000 iterations (plots 202a, 202d, 202g). Approximately 50% of the top 50 novel genes are reported to be associated with diseases in different therapeutic areas (metabolic disorders, immune disease and oncology) after several years (FIGS. 2(a), 2(d) and 2(g) for non-alcoholic fatty liver disease (NAFLD), systemic lupus erythematosus (SLE) and non-small cell lung carcinoma (NSCLC), respectively). Overall, the language model predictions (plots 201a, 201d, 201g in FIGS. 2(a), 2(d) and 2(g), respectively) were 6 to 10 times more likely to be studied than a random sample of genes (plots 202a, 202d, 202g).

The genome-wide rankings from the language models were then evaluated on prioritising therapeutic drug targets before their discovery in a scientific publication and the first clinical trial in FIGS. 2(b), 2(e) and 2(h) for NAFLD, SLE and NSCLC, respectively. The genome-wide rankings for the human protein-coding genes PNPLA3, FGF21, and ANGPTL3 (FIG. 2(b)) rapidly increased before the first publications relating them to NAFLD: PNPLA3 by Romeo in 2008, FGF21 by Inagaki in 2007 and ANGPTL3 by Yilmaz in 2007 (FIG. 2(b)). Furthermore, the genome-wide rankings rapidly prioritised these targets before the first clinical trial with compounds modulating their activity: ION-8391 to inhibit the production of PNPLA3 in 2018, pegbelfermin as a human FGF21 analogue, first reported in 2007, Vupanorsen as an N-acetyl galactosamine-conjugated antisense molecule that targets ANGPTL3 mRNA in 2015 (FIG. 2(b)). Language models are sometimes black boxes, but insights were gained into what drove the predictions by finding ‘contextual linking words’ already published and then prioritising articles containing them. FIG. 2(c) shows the intermediate ‘contextual linking words’ for PNPLA3 and NAFLD. These words include hyperinsulinemia, glucose intolerance, hypertriglyceridemia as terms related to the claims from research performed from 2004 until 2007, that associated PNPLA3 with obesity, insulin resistance in adipose tissues, the phenotypes of NAFLD.

The same applies to the immune-related trait SLE in FIG. 2(e). Bruton tyrosine kinase (BTK) ranked in the top-100 genes from 1999, well before the trial of ibrutinib in 2007. CD19 ranked in the top-300 genes before the first trial of obexelimab in 2007. Both interleukin-1 receptor-associated kinase 4 (IRAK4) and Toll-like receptor 7 (TLR7) were ranked from the top-10000 to the top-1000 genes for SLE before the first publication implicating them in the pathology of lupus, in 2009 by Cohen and 2005 by Barrat, respectively. IRAK4 was consistently ranked in the top-1000 genes prior to the first PF06650833 inhibitor by Pfizer which dates back to 2014. Similarly, TLR7 was on the top ranking for several years before the first TLR7 inhibitor that entered the clinic, IM03100, which was discontinued. Enpatoran is another TLR7 inhibitor currently in Phase 2 trials for lupus. The contextual linking words indicate why the model suggested IRAK4 for lupus, due to its involvement in innate immunity through TLR7 that subsequently activates Myd88 (FIG. 2(f)).

FIGS. 2(g), 2(h) and 2(i) are related to NSCLC. FIG. 2(h) shows that anaplastic lymphoma kinase (ALK) and AXL receptor tyrosine kinase ranked in the top-1000 suggested targets for NSCLC for several years before their first evidence by Soda in 2007 and Wimel in 2001, and the first trials for the ALK inhibitor crizotinib (originally thought as a tyrosine-protein kinase MET specific inhibitor) in 2007 and the AXL inhibitor bemcetinib in 2010. The 2006 language model was aware of their role as receptor tyrosine kinase oncogenes (high similarity to ‘kinase’ and ‘target’ FIG. 2(i) in other cancer indications and their potential implication in NSCLC. The immunomodulatory role of CD274 in tumours was demonstrated by Honjo's group, winner of the Nobel prize in 2002. The language model could not prioritise this gene in the top 1000, but it did before the first monoclonal antibody nivolumab, in 2006. There was prior evidence of the neurotrophic receptor tyrosine kinase 1 (NTRK1) association with types of NSCLC in 1995 but the oncogenic role in lung adenocarcinoma was not confirmed until 2008. NRTK1 ranking was consistently in the top 100 genes (FIG. 2(h)) before Ding results and in the top 20 (FIG. 2(h)) before the first evidence of an NRTK inhibitor, entrecitibib, in 2009. Furthermore, back in 2002, language models were able to prioritise several oncogene targets of NSCLC, genes expressed in lung adenocarcinoma (LUAD, FIG. 2(i)) cell lines (t-test with p-value 1e−198) but not CD274. This suggests that the top-ranking targets are enriched in genes expressed in the tissue of origin of the disease.

The embeddings from 2016 were used as covariate features to estimate the clinical trial success with a multilayer perceptron model. The output generated another score that was tested on prioritising incipient target-disease indications that ended up in clinical trials. On a testing set of 200 diseases described by Paliwal et al., 5.81% of the top 200 targets per disease novel suggestions ended up in a clinical trial after 5 years (FIG. 2(j)). These results outperform the Paliwal et al. method with 3.54% precision for 200 targets per disease. It was found that the method described herein prioritises better (mean z-score is 1.86 in FIG. 2(l)) the 60 targets of SLE that have approved drugs or drugs in active clinical trials in October 2022 in Informa Pharmaprojects. Furthermore, the method disclosed herein gave the highest z-score to the therapeutic targets TLR7, BTK and IRAK4 of which only TLR7 has been reported to have a genetic association with lupus through a single gain-of-function mutation. These results suggest that language models prioritise gene-disease associations before their discovery and validation in clinical trials.

The results shown in FIG. 2 were obtained as follows. Literature associations between genes and diseases were obtained using the approach described in International Patent Publication No. WO 2022/096861 A1. The clinical stages for the target-disease indications were gathered from the manual curation of Informa PharmaProjects and Open Targets datasets. The ‘chembl’ score from Open Targets was recalculated after the integration: a score from 0 to 1 depending on the clinical phase of a target-disease pair: 0.1 for phase 1, 0.2 for phase 2, 0.7 for phase 3 and 1.0 if there is an approved and launched drug. This step scored each target-disease association depending on the clinical trial stage. Gene expression data in non-small cell lung cancer in FIG. 2(i) was obtained from The Cancer Genome Atlas. The threshold was a transcript per million of 0.1 from the lung adenocarcinoma dataset (TCGA-LUAD).

For FIGS. 2J and 2K, multiple feed-forward neural networks with different amounts of dropout, hidden layer sizes and the number of layers were used to regress the ‘chembl’ score from 2016 for FIGS. 2J and 2K. Three hyperparameters were tuned: the number of hidden layers (1 or 2), the dropout (0, 10, 20, 30 or 40%) and the size of the hidden layers (1, 5, 10, 20, 50, 100). The model with the highest validation accuracy on the future (test) set was selected. Embeddings from December 2015 were used as covariate features to regress the ‘chembl’ scores, giving higher scores to approved drugs than to target-disease pairs in phase 1. Data from 2016 was used to demonstrate the ability to prioritise prospective associations until 2022. The training and testing data were split according to ‘Preclinical validation of therapeutic targets predicted by tensor factorization on heterogeneous graphs’, Paliwal et al., Sci. Rep. 10, 18250 (2020): 200 diseases and all their targets were in the testing set, and the remaining diseases and their targets were part of the training set. Only 40 out of the 200 diseases were released in Supplementary Table 6 (Paliwal et al.), and 160 were randomly selected to contain a mix of metabolic, immune, neurological and oncological indications.

For FIG. 2(i), genome-wide summary statistics to run the MAGENTA pipeline (‘Common Inherited Variation in Mitochondrial Genes Is Not Enriched for Associations with Type 2 Diabetes or Related Glycemic Traits’, Segré et al., PLOS Genet. 6, e1001058 (2010)) were obtained from ‘Identification of 38 novel loci for systemic lupus erythematosus and genetic heterogeneity between ancestral groups’, Wang et al., Nat. Commun. 12, 772 (2021). MAGENTA ran with default parameters with the summary statistics and all data in the genome build Genome Reference Consortium 37 (GRCh37). To run the Priority index (Pi in FIG. 2(i)), we used a genome-wide summary statistics pipeline from Open Targets Genetics. A total of 15,901 genes are prioritised, based on 6645 single nucleotide polymorphisms (SNPs) scored positively (including 644 ‘Lead’ and 6001 ‘Linkage Disequilibrium’ genes); 714 nearby genes within 50,000 base pairs genomic distance window of 6129 SNPs 2045 expression Quantitative Trait Loci (eQTL) genes with expression modulated by 4677 SNPs; 912 HiC genes physically interacted with 4033 SNP; 2900 genes defined as seeds from 6645 SNPs; randomly walk the network (15901 nodes and 318866 edges from the STRING database) starting from 2900 seed genes with a restarting probability of 0.75. The Open Targets dataset only gives an overall score between 0 and 1 to targets with evidence for diseases. To generate a genome-wide ranking to calculate the z-scores, the remaining non-associated targets were given a random score (uniform distribution) between the minimum association score for the disease and zero. Z-scores were calculated with the quantile normalisation function in Scikit Learn.

The list of therapeutic targets in this section were downloaded from Pharmaprojects and included all therapeutic targets for systemic lupus erythematosus that had an active program in the following stages: Phase I Clinical Trial, Phase II Clinical Trial, Phase Ill Clinical Trial, Registered, and Launched. This included the following gene symbols: BTK, BTLA, CD200, CD200R1, CD22, CD28, CD38, CD40, CD40LG, CD6, CD79B, CLEC4C, CNR2, CRBN, CXCR5, FCGR2B, FCGRT, FKBP1A, ICOSLG, IFNA1, IFNAR1, IKBKE, IL12B, IL1RL2, IL2, IL21, IL23R, IL2RA, IL2RB, IL2RG, IRAK1, IRAK4, JAK1, JAK3, LANCL2, LGALS1, LGALS3, LILRA4, MALT1, MASP2, MC2R, MIF, MS4A1, NLRP3, NR3C1, PIK3CB, S1PR1, SIK2, SLC15A4, SNRNP70, SOCS1, STING1, SYK, TBK1, TLR7, TLR8, TNFRSF13B, TNFRSF13C, TNFRSF4, TNFSF13, TNFSF13B, TYK2, XPO1.

### Example 2

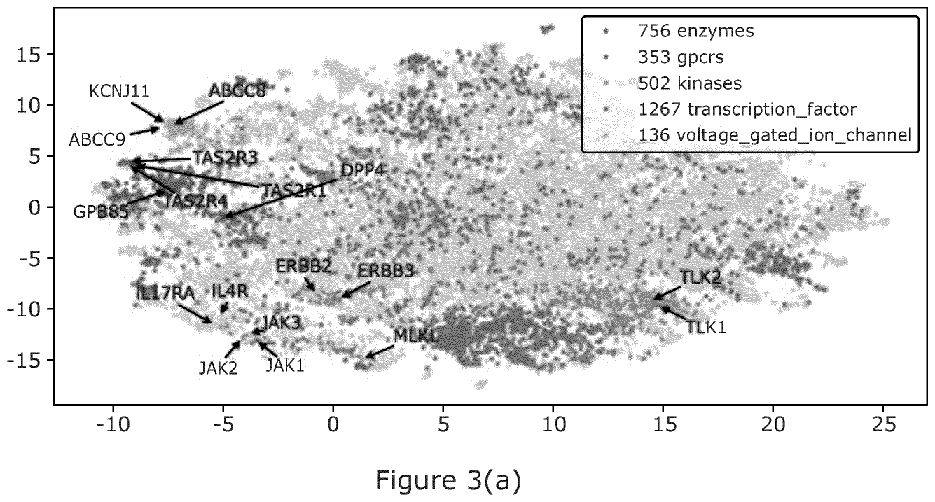

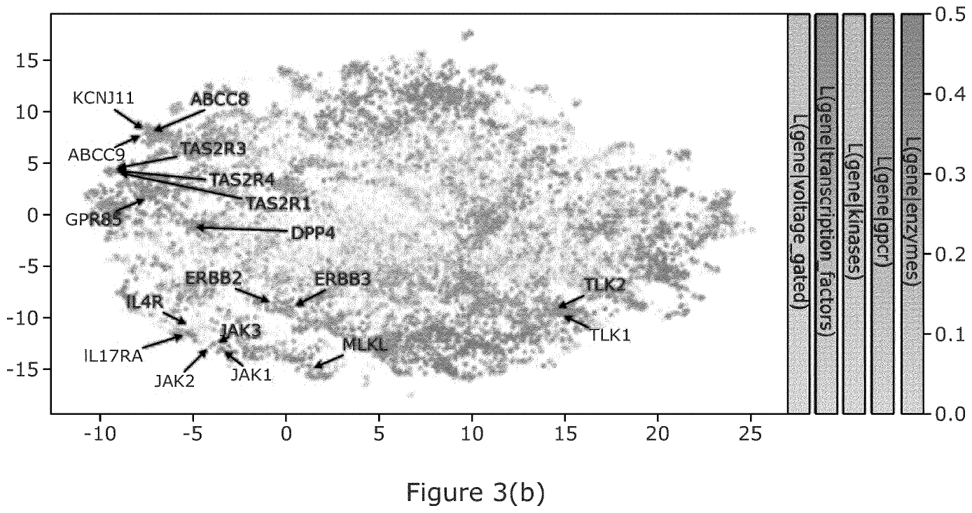

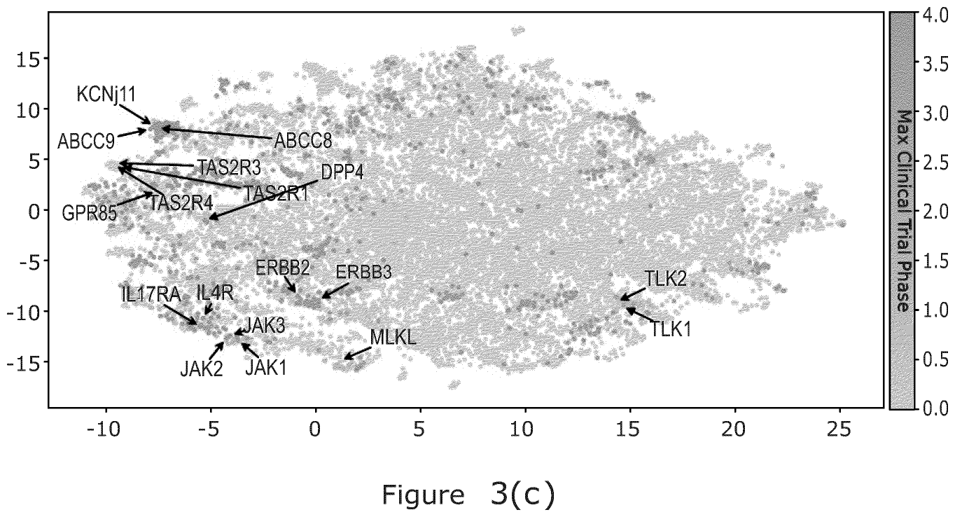

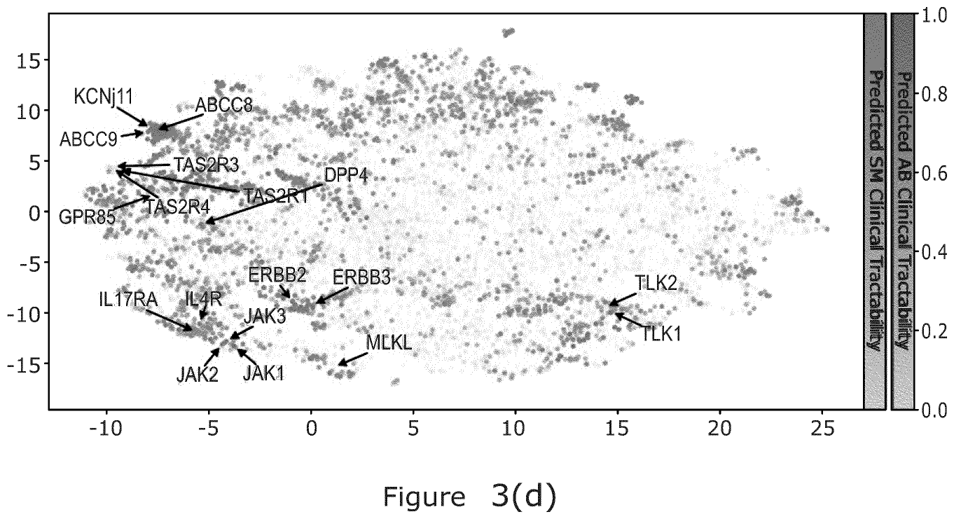

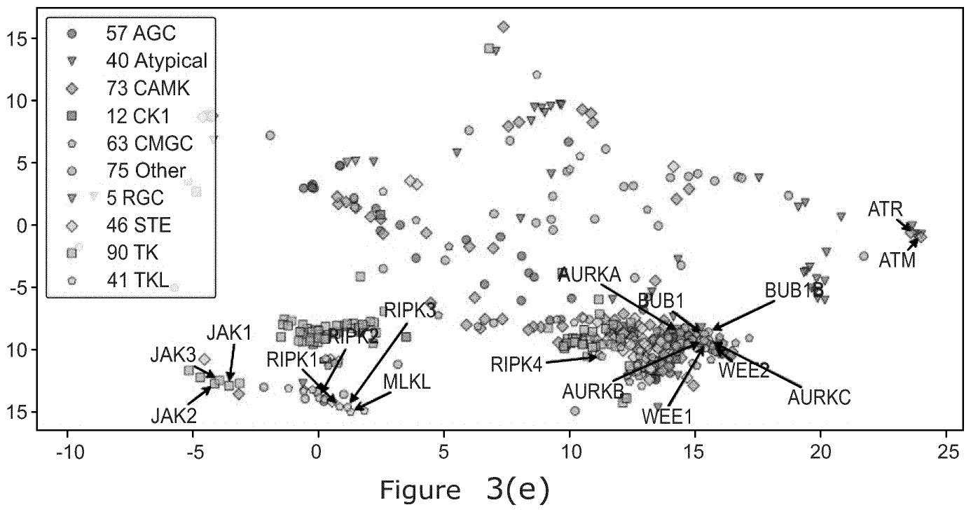

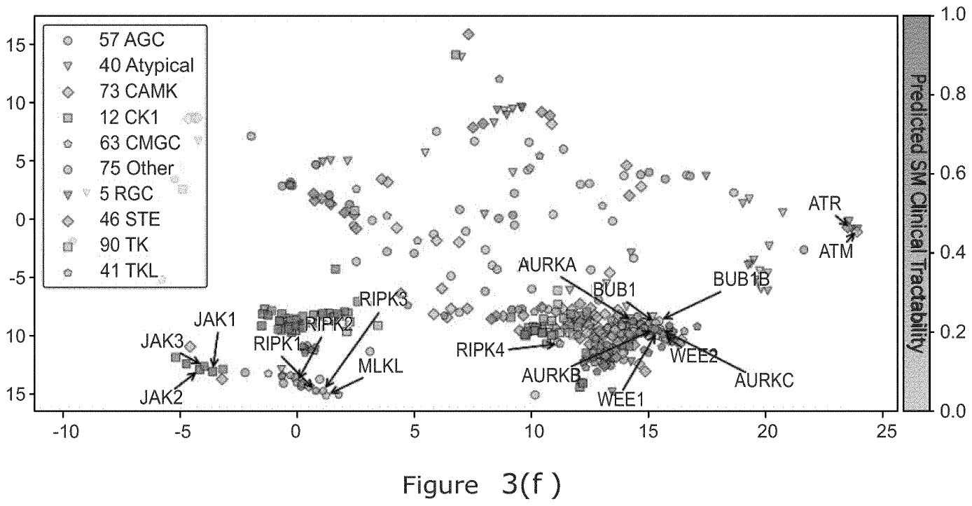

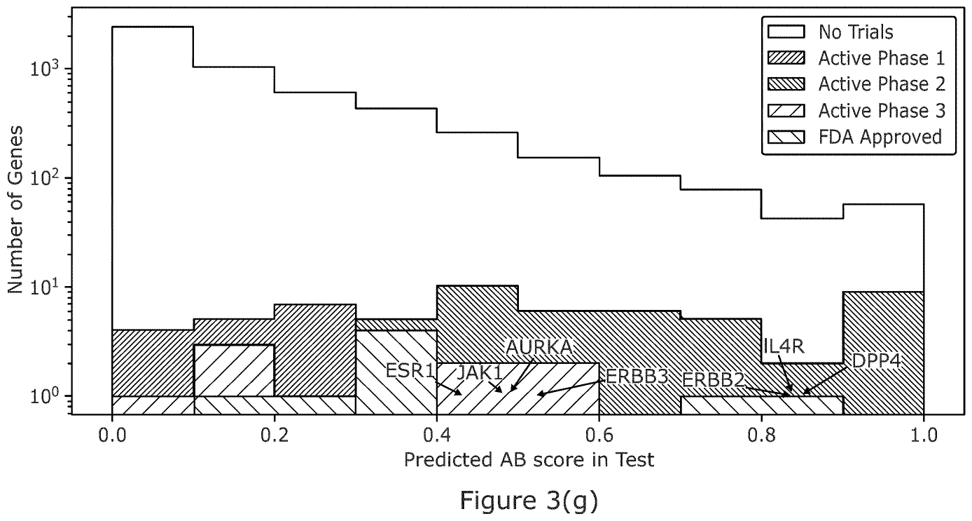

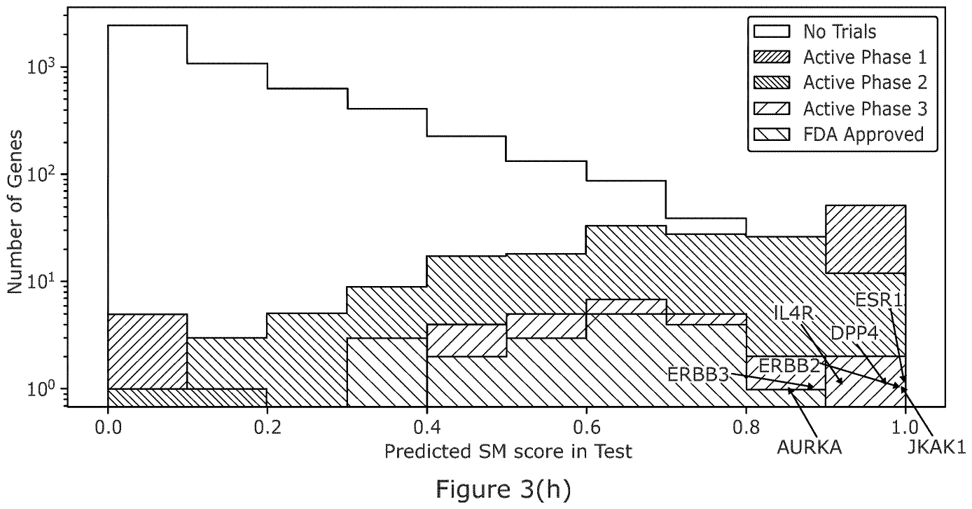

In another example, an exploratory analysis was conducted with the 2022 model to understand how one biomedical entity, human genes, were represented in a low dimensional space. FIG. 3 illustrates results of a Uniform Manifold Approximation and Projection (UMAP) of the Word2Vec embeddings for the human protein-coding, published genome (n=19,229 data points). In FIG. 3(a), several drug therapeutic target classes are visualised in the lower dimensional representation with the first two UMAP components (enzymes, g-protein-coupled receptors, ion channels, kinases) and transcription factors considered undruggable until the advent of PROTACs. FIG. 3(b) illustrates UMAP lower dimensional representation shaded by word co-occurrence likelihoods of the gene tokens and several tokens related to the therapeutic target classes (enzyme, GPCRs, voltage gated, kinases, transcription_factors) highlighting the same areas as FIG. 3(a). FIG. 3(c) illustrates the same UMAP representations shading the maximum achieved clinical trial phase according to PharmaProjects. FIG. 3(d) shows predicted small molecule (SM) and monoclonal antibody (AB) estimates using a multi-task logistic regression classifier that takes the human gene embeddings as input. The gene embeddings can be thought of as fingerprints for downstream estimation tasks. FIG. 3(e) shows a subset of genes that encode human kinases coloured by the different kinase families. The meaning of the kinase family acronyms are AGC for kinase A, G, and C families (PKA, PKC, PKG), CAMK for calmodulin-dependent protein kinase, CK1 for casein kinases, CMGC for cyclin-dependent kinases and mitogen-activated protein kinases and glycogen synthase kinases and CDK-like kinases, RGC for receptor guanylate cyclases, STE for serine/threonine kinases, TK for tyrosine kinase, TKL for tyrosine kinase-like. FIG. 3(f) shows the same plot as FIG. 3(e) but shaded based on the small molecule predicted clinical tractability.

The Uniform Manifold Approximation and Projection (UMAP) revealed some hidden structure of the embeddings in the low dimensional space (FIGS. 3(a) to 3(f)). The different therapeutic drug target families are clustered in different regions of the space (FIG. 3(a)). The mean Silhouette Coefficient was 0.11 (out of a maximum of 1), a measure of how consistent the gene families were in the lower dimensional space. The mean cosine distance between all human genes was 0.14, whereas it was 0.19 between the enzymes, 0.28 for G-protein coupled receptors (GPCRs), 0.27 between the kinases, 0.23 between the transcription factors and 0.38 for the voltage-gated ion channels. The cosine distance is a measure of similarity between two vectors or embeddings. These gene family clusters in FIG. 3(a) overlap when the human genes are shaded based on the learnt likelihood to co-occur with phrases that describe the gene families (FIG. 3(b)). Regarding individual genes, the gene embeddings cluster voltage-gated ion-channels genes such as ABCC8, ABCC9 and KCNJ11, the three are targets of type 2 diabetes mellitus; JAK1, JAK2 and JAK3 kinases that are therapeutic targets in many immune-related disorders; and the receptor tyrosine kinases ERBB2 and ERBB3. Furthermore, therapeutic drug targets with programs in clinical trials or with drugs approved appear in the periphery of the UMAP plot in FIG. 3(c). These vector embeddings contain the information from the literature compressed as dense fingerprints and they can be used in subsequent classification tasks.

To test whether the embeddings contained information on successful clinical trial targets, the classification experiment from ‘In silico prediction of novel therapeutic targets using gene-disease association data’, Ferrero et al., J. Transl. Med. 15, 182 (2017) was repeated. Ferrero et al. used several manually engineered features from the Open Targets platform to predict whether a gene was or was not a therapeutic target. In this test, the features were the word vector embeddings trained until December 2017. The model was a multilayer perceptron with default parameters and a balanced loss function. Genes were labelled as targets if Informa PharmaProjects tagged them in any active clinical trial or as registered or launched in 2017, as Ferrero et al. did. The most advanced stage was considered for targets with programmes across phases. The choice of 2017 was to directly compare to the state of the literature and the clinical trial phases when the Ferrero et al. research was carried out. Table 2 (below) contains the classification metrics for the testing set with unseen genes to a random forest classifier. These metrics were calculated with a comparable number of negatives as positives. The numbers in brackets represent the 5% and 95% confidence intervals from 100 bootstrap sampling experiments with a balanced number of positive and negative samples. Precision is the fraction of actual targets versus all predicted targets. Recall stands for the fraction of predicted targets compared to the number of test targets. The f1 score is a metric that averages the precision and the recall. The f1 score (Table 2, Approved or Clinical Trial column) is 20% higher than reported by Ferrero et al. Open Targets integrates manually engineered variables that define a gene as a target from multiple sources. These results suggest that word vectors capture more about therapeutic targets than variables handcrafted by experts. Classifiers could be used to generate a learned clinical tractability estimate.

TABLE 2

| Classifi- | | | |
|---|---|---|---|
| cation | Target in | Target has an | Approved or |
| Metric | Clinical Trial | Approved Drug | Clinical Trial |
| Accu- | 0.719 [0.665, 0.849] | 0.801 [0.76, 0.841] | 0.877 [0.822, 0.908] |
| racy | | | |
| score | | | |
| F1 | 0.618 [0.558, 0.673] | 0.667 [0.602, 0.697] | 0.875 [0.808, 0.993] |
| score | | | |
| Preci- | 0.498 [0.431, 0.531] | 0.543 [0.467, 0.574] | 0.889 [0.838, 0.932] |
| sion | | | |
| Recall | 0.816 [0.712, 0.848] | 0.864 [0.792, 0.917] | 0.862 [0.829, 0.938] |

The classification experiment was repeated separately for small molecule (SM) and monoclonal antibody (AB) targets (FIG. 3(g)). The predicted SM clinical tractability probabilities are shown in FIG. 3(d) for the entire genome and FIG. 3(f) only for all human kinase genes. Despite the fact that there are no monoclonal antibodies against the known type-2 diabetes small molecule targets ABCC8, ABCC9, DPP4 and KCNJ11, these genes had AB tractability estimates higher than 60% as well as the interleukin receptors IL4R and IL17RA (FIG. 3(d)). These genes are transmembrane proteins, amenable to monoclonal antibody targeting, and the language model correctly captured this information despite evidence in the training set. Similar results were obtained for ERBB2 and ERBB3, with 90% SM and AB scores, and FDA approved small molecules and antibodies against them. FIG. 3(e) contains only the 502 kinases (FIG. 3(a) representing all 16,533 genes) represented in the lower dimension. In FIGS. 3(e) and 3(f), genes that encode protein kinases cluster based on their biological pathways and their functions are indicated. Receptor-interacting serine/threonine-protein kinases RIPK1, RIPK2, RIPK3 and MLKL involved in necroptosis, the Janus tyrosine kinases JAK1, JAK2 and JAK3 cluster on the bottom centre of FIG. 3(e). Furthermore, the kinases involved in DNA damage repair also cluster together in FIG. 3(e). Regarding the small molecule tractability estimates, MLKL, TLK1 and TLK2 receive very low scores as they are kinase-like proteins that have lost their kinase activity. The kinase families on the legend are the acronyms used by Guide to Pharmacology. These results suggest that language embeddings contain features useful to define therapeutic drug targets. However, the crucial step in drug discovery is identifying the right therapeutic drug target for the right disease.

The results illustrated in FIG. 3 were obtained as follows. The clinical stages for the targets were gathered from Informa PharmaProjects and Open Targets datasets. The therapeutic target classes and the kinase families were downloaded from the Guide to Pharmacology database. The Uniform Manifold Approximation and Projection (UMAP) low dimensional representation was achieved using the umap-learn package in Python with the following hyperparameters: 15 neighbours, cosine similarity as the metric, 1000 epochs, a repulsion strength of 12 and local connectivity of 3 neighbours. To measure the consistency of the clusters in FIG. 3(a), the differences in the cosine distances were compared. The cosine distance represents the angle between two vectors. The more similar the vectors, the closer the angle to 1. The intercluster cosine distance was significant for all gene families with the following P-values using a t-test with unequal variance and a nonparametric Mann-Whitney U test: 3.03e−251 and 7.49e−249 for enzymes, 3.17e−321 and 0.0 for G-protein coupled receptors, 2.45e−304 and 6.96e−166 for kinases, 4.38e−124 and 3.51e−46 for voltage-gated ion channels.

The same hyperparameters as in ‘In silico prediction of novel therapeutic targets using gene-disease association data’, Ferrero et al., J. Transl. Med. 15, 182 (2017) were used for the multilayer perceptron, a feed-forward neural network with a single layer and a balanced function implemented in SciKit Learn. The solver was Adam, and the network stopped if the performance did not improve for two epochs in a validation set. The input data was the gene embeddings from the language model from December 2016 to directly compare to Open Targets features at the time. The metrics displayed in Table 2 correspond to an independent, balanced test set. The same model architecture was used to classify a gene into a tractable target by monoclonal antibodies and small molecules in FIGS. 2(g) and 2(h). The data for the labels for whether a target is tractable with small molecules or antibodies was gathered from manual curation of Informa PharmaProjects and Open Targets. The datasets in this section were randomly split into training and test sets, containing 80 and 20% of the observations (similar to Ferrero et al.), respectively.

### Example 3

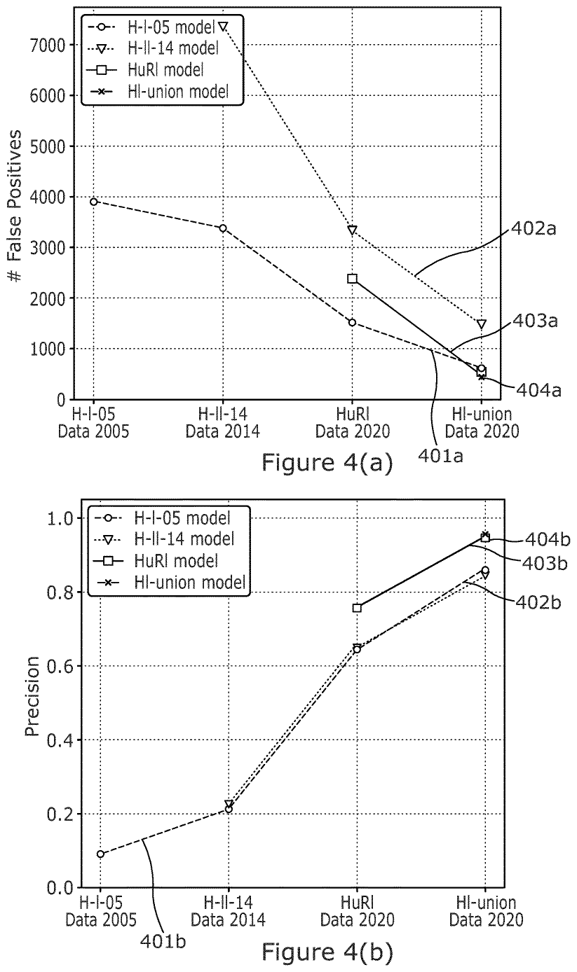

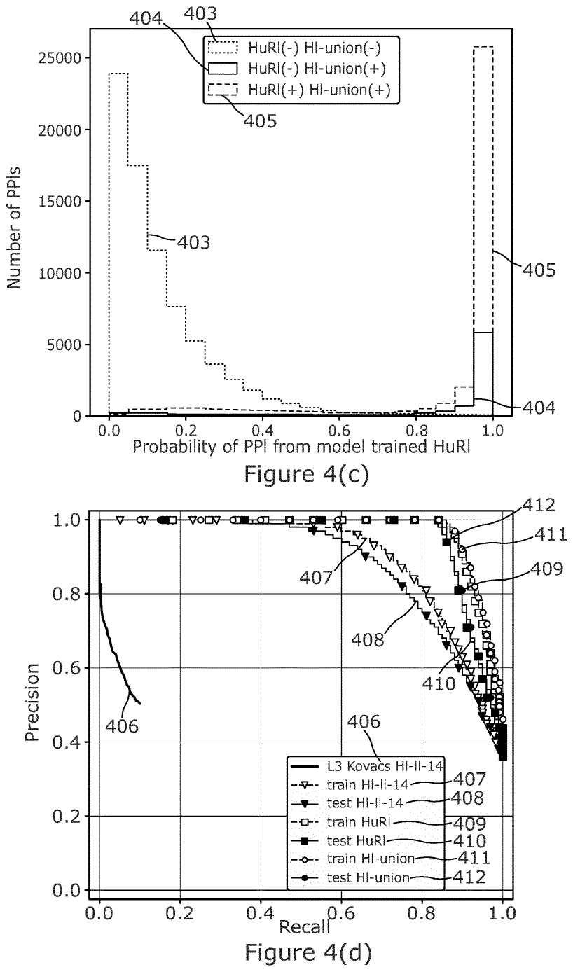

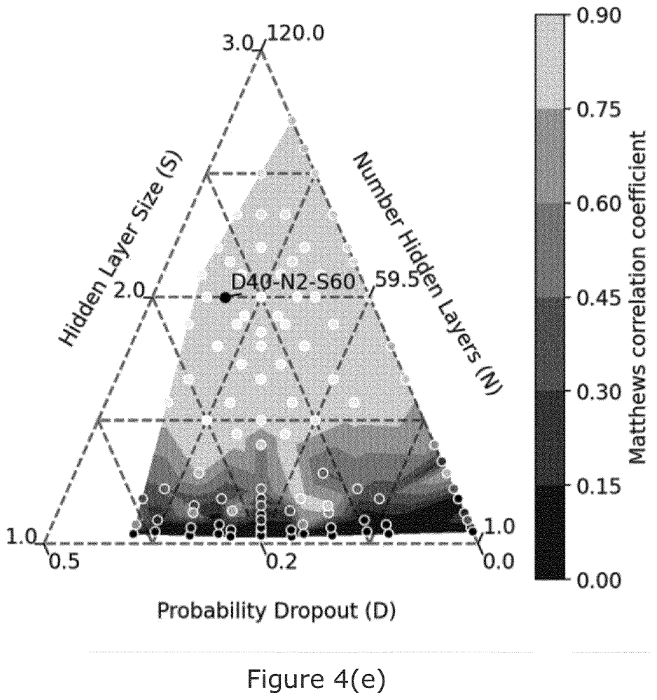

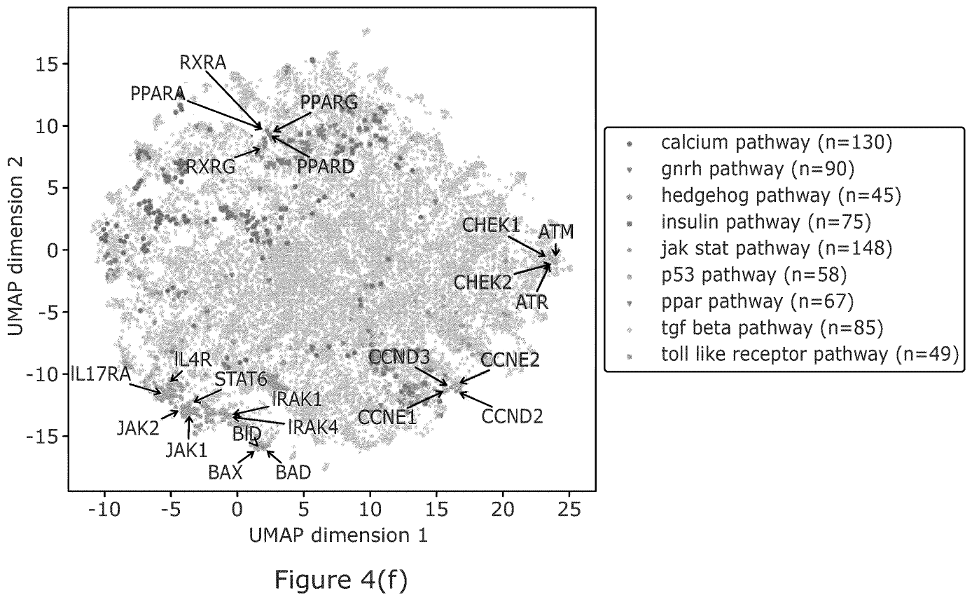

In another example, the described method was used to prioritise protein-protein interactions. FIGS. 4(a) and 4(b) show plots containing the historical prediction accuracies for the Human Interactome. The number of false positives is reported in FIG. 4(a) and the precision over time in FIG. 4(b) for the different historical datasets (H-I-05 labelled 401a, 401b, HI-II-14 labelled 402a, 402b, HuRI labelled 403a, 403b and HI-union labelled 404a, 404b) only for the testing set. Earlier models are evaluated on newer datasets; the H-I-05 model was trained on H-I-05 positive examples and evaluated on later datasets. FIG. 4(c) shows a histogram of the predicted score for the positive, not-yet-positive and negative sets of HuRI and HI-union. Histogram plot of the scores from the model trained on HuRI positive examples for the HuRI examples is on the x-axis versus the number of protein-protein interactions on the y-axis. HuRI negatives are labelled 403, HuRI positives are labelled 405, and HuRI negatives and HI-union positives are labelled 404. The HuRI model had an area-under-the-curve (AUC) score of 86.6% on the HuRI dataset and 89.7% on the HI-union dataset. In FIG. 4(d), it is shown that the described method outperforms the L3 method in ‘Network-based prediction of protein interactions’, Kovacs et al., Nat. Commun. 10, 1240 (2019) (mean cross-validation labelled 406) in all cases. 25% of the genes and all their interactions were used in the test set. The remaining interactions were used as the input network to predict the remaining protein-protein interactions. FIG. 4(e) shows a contour ternary plot for 3 hyper-parameters and the Matthew correlation coefficient (MCC) on the HI-union dataset. Three hyperparameters tuned: hidden layer size ‘S’, number of hidden layers ‘H’, and the dropout probability ‘D’. Higher MMC values have lighter shading, meaning a better model on the unseen 25% of the genes and all their pairs in the testing set. The best model with a dropout of 40%, 2 layers and 60 units is highlighted in black shading with an MCC score of 89%. In FIG. 4(d), UMAP coordinates (same as in FIGS. 2(a) to 2(d)) and shaded based on pathways from the Kyoto Encyclopedia of Genes and Genomes (KEGG): ‘calcium pathway’ with 130 genes, ‘Gonadotropin-releasing hormone pathway’ (GnRH) (n=90), ‘insulin pathway’ (n=75), Peroxisome proliferator-activated receptors (PPAR) pathway (n=67), ‘transforming growth factor beta (TGFB) pathway’ (n=85) and ‘Toll-like receptor pathway’ (n=49).

The incomplete interactome limits our ability to understand the molecular roots of human diseases. To infer plausible targets, the pathways affected by the disease need to be known to select a node in the signalling network that, if modulated, palliates the disease state. It was tested whether the described language models prioritise protein-protein interactions before publication. The historical data for the protein-protein interactions was gathered from yeast-two-hybrid assays from the human interactome. The human interactome contains four historical datasets: H-I-05 in 2005, HI-II-14 in 2014, HuRI in 2020 and HI-union in 2020. There was no bona fide negative set. Combinations of ‘sticky’ proteins, the top 10% of proteins with most interactions, were negative examples unless they intersected with the positive set. To test the ability of the models to correctly predict true protein-protein interactions eventually discovered, new data was regarded as negative observations for older models, and predictions were evaluated on the same year and prospectively.

The prospective analysis showed that the number of false positives always decayed in each prospective dataset (FIG. 4(a)), and precision always rose (FIG. 4(b)), where H-I-05 is labelled 401a, 401b, HI-II-14 is labelled 402a, 402b, HuRI is labelled 403a, 403b and HI-union is labelled 404a, 404b. The difference in false positives between subsequent datasets corresponds to the number of protein-protein interactions that could have been prioritised 6 to 9 years ahead of time (FIG. 4(a)). In some cases, this number of correctly predicted protein-protein interactions ranges between 500 and 2000. Eventual true positives (e.g. 2014 and 2020) not yet discovered in 2005 counted as negative samples in the training set, and they were still prioritised due to the positive-unlabelled learning strategy (FIGS. 4(a) and 4(b)). As an example, most HI-union positive but HuRI negative protein interacting pairs received probability scores (HuRI(−) HI-union(+) 404 in FIG. 4(c)). These high scores correspond to the decrease in the number of false positives in FIG. 4(a) for the HuRI model and the rise in precision in FIG. 4(b) for the same model. The described method outperformed the L3 method in ‘Network-based prediction of protein interactions’, Kovacs et al., Nat. Commun. 10, 1240 (2019) at different thresholds in a precision-recall curve in both the training and testing set (FIG. 4(d)). There was a slight overfitting in the training set (FIG. 4(d)) but the models were able to generalise to unseen proteins as the test set. Sixty multilayer perceptron models (one dot per model in contour plot of FIG. 4(e)) were trained with three different hyper-parameters: number of hidden layers, hidden layer size, and dropout percentage. The models need a relatively large hidden layer size (>10) to correctly classify protein-protein interactions (Matthews correlation coefficient or MCC>0.8) on the test set (FIG. 4(e)). The best model was the D40-N2-S60 with 40% dropout, two hidden layers and 60 units on the HI-union dataset. The multilayer perceptron models that used embedding features could correctly predict protein-protein interactions because the embeddings encode information about the biochemical pathways in which the genes are involved (FIG. 4(f)). Members of the peroxisome proliferator-activated receptors (PPAR) pathway with 67 genes involved (including PPARA, PPARD, PPARG, RCRA, RXRG in FIG. 4(f)) from Kyoto Encyclopedia of Genes and Genomes (KEGG) cluster together (mean cosine distance of 0.32 versus a 0.14 for any-to-any gene FIG. 4(f)). As another example, members of the JAK-signal transducer and activator of transcription (STAT) pathway with 148 genes involved (including JAK1, JAK2, STAT6, IL4R, IL17RA in FIG. 4(f)) from KEGG cluster together (mean cosine distance of 0.31 versus a 0.14 for any-to-any gene FIG. 4(f)). The multilayer perceptron model trained with embeddings outperforms the L3 method (labelled 406 in FIG. 4(d)) and could help to prioritise low throughput experimental validation of protein-protein interactions.

The results illustrated in FIG. 4 were obtained as follows. All historical versions of the human interactome were used, accounting for 64,000 human protein-protein interactions in its latest release. Self-interactions were discarded like self-phosphorylations in tyrosine kinase receptors. Protein interaction pairs were augmented by reversing the gene order (e.g. ‘A1BG-ZNF44’=>‘ZNF44-A1 BG’). These interactions were positive examples. There was no bona fide negative set. Therefore, all combinations of the top 10% of proteins with most interactions were negative examples unless they intersected with the positive set. For example, even though the genes DVL2 and TRAF2 had 36 and 69 interaction pairs, the DVL2-TRAF2 pair was excluded from the negative pair set because it was in the H-I-05 positive pair set. However, the pair DVL2-NIF3L1, with 36 and 31 interactions respectively in H-I-05, was included in the set of negative pairs.

To test the ability of the models to eventually recover true protein-protein interactions, the newly positive interactions in 2020 were regarded as negative examples to the 2014 and 2005 models. For example, the CCHCR1-ZWINT interaction introduced in HuRI was a positive example for the 2020 experiment but negative for the 2014 and 2005 experiments. The idea was to test whether a positive-unlabelled strategy could prioritise future protein interaction pairs even if they were presented as negatives. The features were the concatenation of the two embeddings for the proteins in the pair. Language models were checkpointed each December. GPT2 embeddings trained until December 2005 were the features for the model trained on H-I-05 examples, GPT2 Dec. 2014 embeddings to train with HI-II-14 positive examples and GPT2 Dec. 2020 embeddings with positive examples from HuRI and HI-union.

The split into training, validation and test sets was done in two ways. There was a protein-based split for testing. A random split of 1:3 for the test and train sets at the protein but not the pair level. 25% of genes and all their partners are just on the test set and never seen during the training. Also, there was a time-based split for validation. The predictions were also evaluated prospectively in FIGS. 4(a), 4(b) and 4(c). Language models were trained up to different dates in the past. Neural network models with past embeddings were trained with old protein interaction pairs and evaluated on newer datasets. For example, the H-1-05 multi-layer perceptron was trained with 2005 embeddings and H-I-05 examples. This H-I-05 multi-layer perceptron was evaluated on H-I-05 interaction pairs and prospective data: HI-II-14 and HuRI and HI-union (FIGS. 4(a), 4(b) and 4(c)).

A grid search was performed for the hyperparameter tuning. Three hyperparameters were tuned: number of hidden layers (1 or 2), the dropout (0, 10, 20, 30 or 40%) and the size of the hidden layers (1, 5, 10, 20, 50, 100). This tuning generated 60 different models with different combinations of hyperparameters that resulted in the contour ternary plot in FIG. 4(e).

### Example 4

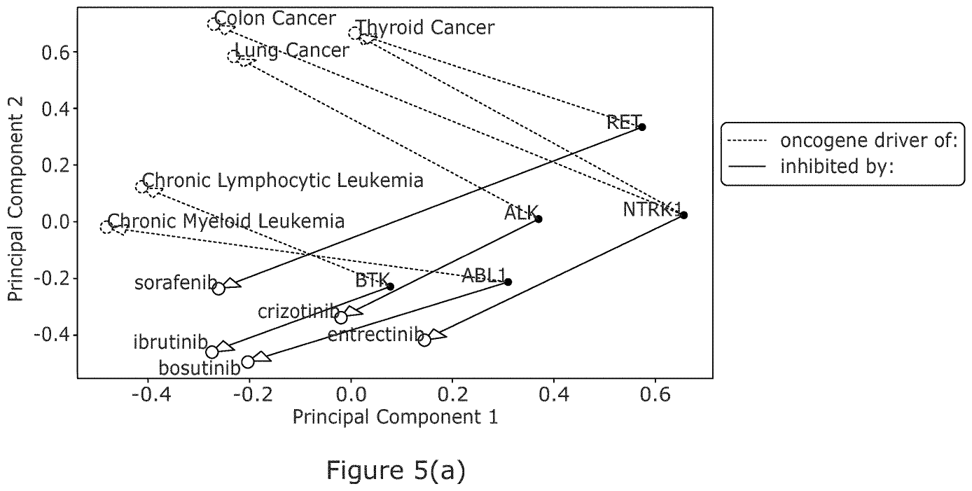

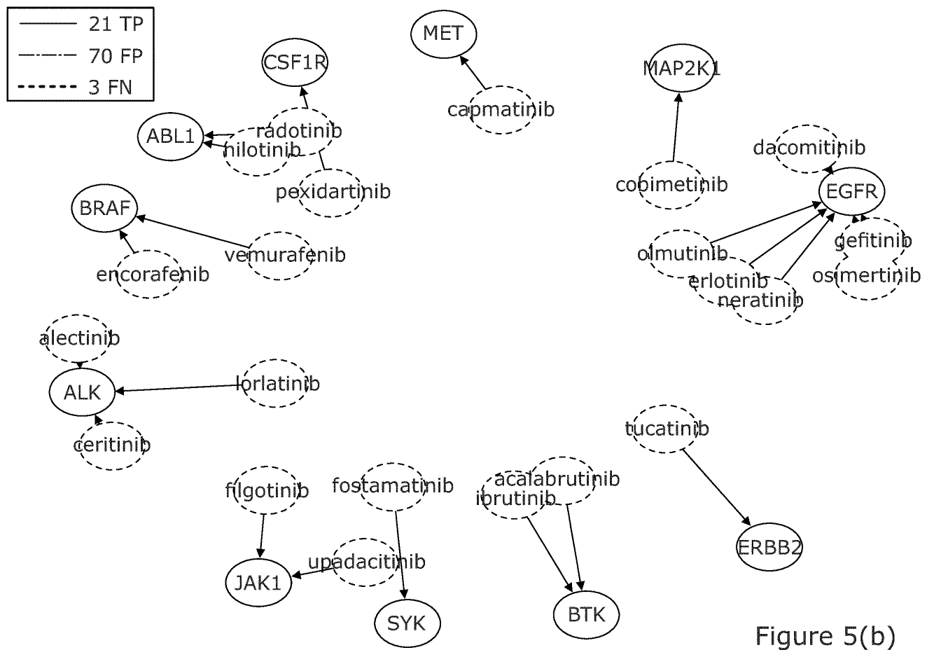

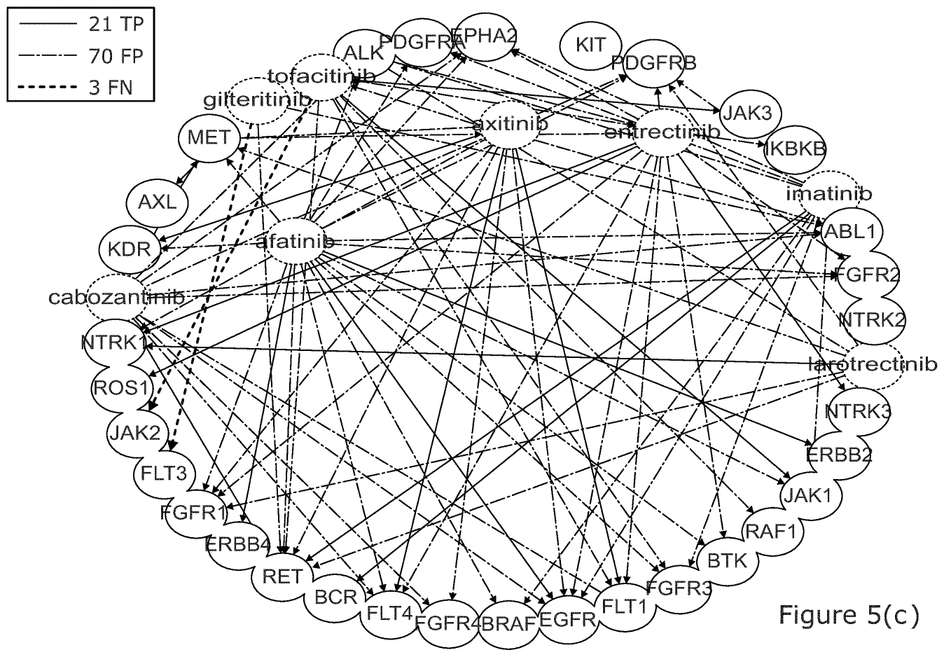

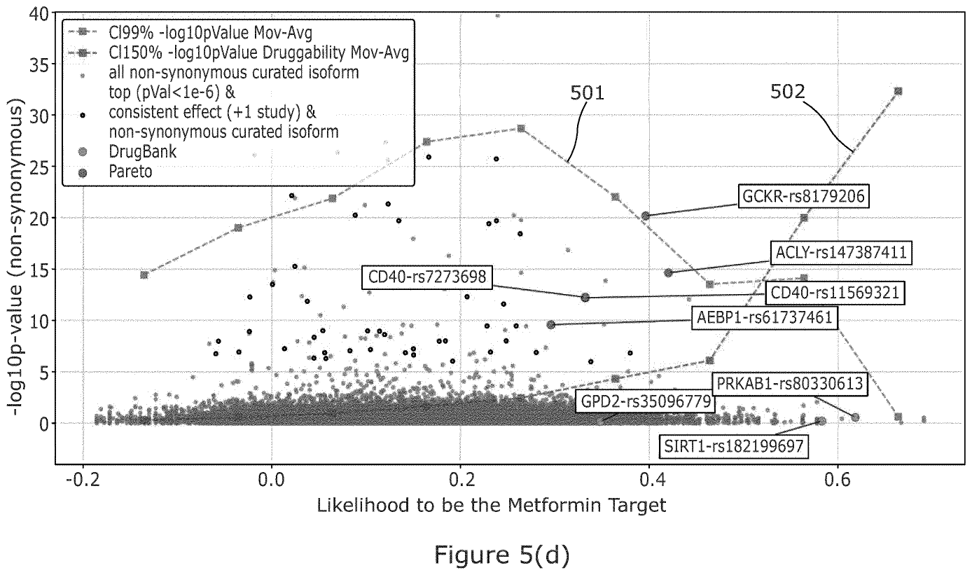

In another example, the described method was used for prioritising novel mechanisms of action of drugs. FIG. 5(a) shows latent relationships learnt between kinase inhibitors, oncogene drivers and cancers. The embeddings for cancer indications, oncogenic tyrosine kinases and kinase inhibitors were projected onto two dimensions using Principal Component Analysis (PCA). There are consistent vector operations between words representing ‘kinase inhibitor of’ and ‘oncogenic gain of function in cancer’. FIGS. 5(b) and 5(c) show predicted links between kinase and kinase inhibitors. The predictions were obtained from the best model called H100-N2-D0.2. Network of kinases (darker shading), kinase inhibitors (lighter shading) and their links (true positives) and link predictions (true positives and false positives): true positives in darker shading, false positives in medium shading, and false negatives in lighter shading. All DrugBank positive links are plotted, and the predicted links with probabilities higher than 0.65. Node size is proportional to the out-degree of each node. The width of the links is proportional to the predicted probability. FIG. 5(b) shows a plots of specific drugs with a maximum of two targets. There are no false positives or false negatives. FIG. 5(c) shows a plot of promiscuous drugs. There are 102 false positives among the drugs and kinases plotted in the graph. FIG. 5(d) shows target prioritisation plot for metformin. Scatter plot for the likelihood (x-axis) to be the metformin target and the −log 10 p-values (y-axis) from the meta-GWAS for non-synonymous variants. The putative targets of metformin SIRT1, AMPK subunit and the mitochondrial glycerophosphate dehydrogenase (GPD2) are indicated. Non-synonymous variants with p-values <10-6 and two studies with the same direction of the allele effect are also indicated. The non-synonymous variants with high likelihoods and genome-wide significance (p-value <10e−8) are indicated. Two lines were calculated with a range interval moving average from left to right. The 99% confidence interval p-value significance from the GWAS meta-analysis is plotted and labelled 501. The moving average for druggability, as defined in ‘A multilayer network approach for guiding drug repositioning in neglected diseases’ Berenstein et al., PLoS Negl. Trop. Dis. 10, e0004300 (2016) is plotted and labelled 502.

Table 3 (below) shows classification accuracies grouped by promiscuous drugs. Classification statistics for predicting links from compounds whose pharmacological mechanism of action is known or at least reported in DrugBank and their protein targets. The results presented in this table are only for the testing set unseen during training. The table is grouped by the number of reported protein targets in DrugBank (more than 2, or 2 or less).

TABLE 3

| | | Pharma- | | Pharma- |
|---|---|---|---|---|
| | No active | cologically | No active | cologically |
| | (Num. | active (Num. | (Num. | active (Num. |
| Metric | Targets >2) | Targets >2) | Targets ≤2) | Targets ≤2) |
| Accuracy score | 0.835 | 0.835 | 0.899 | 0.899 |
| F1 score | 0.907 | 0.221 | 0.878 | 0.913 |
| Precision score | 0.995 | 0.127 | 0.894 | 0.902 |
| Recall score | 0.834 | 0.856 | 0.863 | 0.925 |

Table 4 (below) shows top-ranking genes. Gene symbol, allele change location in the Genome Reference Contig GRCh37, amino acid replacement, reference single nucleotide polymorphism (SNP) identifier, beta coefficient, −log 10 of the p-value, the number of independent studies from the meta GWAS, number of samples as the sum of each study, and the direction of the effects: the sign of the beta coefficient (minus sign for negative, plus sign for positive coefficients, interrogation sign for variants not measured and coefficients not calculated). Genes in this table were non-synonymous, had a p-value lower than 10e−9, had two studies with the same direction in the beta coefficient, and had a high likelihood of being the target of metformin according to the language model.

TABLE 4

| | | | | | | Number | |
|---|---|---|---|---|---|---|---|
| | | Allele | Amino Acid | Beta | −log10 | meta | |
| Gene | Variant | Frequency | Change | Coefficient | p-value | GWAS | Samples |
| ACLY | rs147387411 | <0.001 | Arg582Gln | −3.438 | 14.644 | 2 | 7048 |
| AEBP1 | rs61737461 | <0.001 | Ile444Leu | −1.595 | 9.589 | 2 | 7048 |
| CD40 | rs11569321 | <0.001 | Ser124Leu | −1.673 | 12.21 | 2 | 7048 |
| CD40 | rs7273698 | <0.001 | Phe150Phe | −1.675 | 12.261 | 2 | 7048 |
| GCKR | rs8179206 | <0.001 | Glu77Gly | −2.945 | 20.18 | 2 | 7048 |

Similar to gene-disease associations (FIG. 5(a) kinase to cancer indication), language models also learn latent associations between proteins and the drug-like compounds that modulate their activity (FIG. 5(b) kinase to kinase inhibitor). Even in the reduced dimensions of a principal component analysis, there is a consistent vector operation of ‘kinase inhibitor’+‘kinase inhibitor of’=‘kinase’ and ‘kinase’+‘gain of function in’=‘cancer’ (FIG. 5(a)).

Multiple Scikit Learn and TensorFlow models were trained with the gene and compound word vectors as input, learning from positive samples (protein to compound pairs) from Drugbank and negative random pairs. The vectors for the genes were concatenated with the vectors for the compounds, generating a single fingerprint for each target-compound pair. The models were used to weight the features and yield a single score for each gene-compound pair, that estimates the likelihood that the compound modulates the activity of the protein encoded by the gene. The best performance was achieved by the H100-N2-D0.2 model, a multilayer perceptron classifier. This model had 100 hidden neurons (H100), 2 hidden layers (N2) and 0.2 of dropout (D0.2). The precision for this model was relatively low. Only 17% of the true positives were correctly predicted with high probability over the entire set of positives (Table 3). However, there was a differential performance when the results were grouped by the specificity of the drug-like compounds. The precision increased from 17% to 90% for selective compounds that only act on one or two human proteins. The H100-N2-D0.2 model had a low specificity (12%) for promiscuous compounds that act on three or more targets (Table 3), such as unspecific kinase inhibitors that may act on multiple kinases (FIG. 5(c)). The H100-N2-D0.2 multilayer perceptron classifier correctly assigned high scores to the targets of selective kinase inhibitors exemplified by vemurafenib and encorafenib for BRAF, osimertinib, erlotinib, gefitinib and neratinib for EGFR, and ibrutinib and acalabrutinib for BTK (FIG. 5(b)).

As the language models were able to prioritise known mechanisms of action of drug-like compounds as shown in Table 3, the models were tested on drugs with unresolved targets. Metformin is a widely-used drug to improve glucose metabolism and diabetes-related complications. Several metformin targets have been described, including AMPK, the mitochondrial respiratory chain complex 1 and the mitochondrial GPD2. However, the exact mechanism of action for metformin is still not fully understood. The genome-wide target ranking was checked for metformin and compared with genome-wide association data for glycaemic response (FIG. 5(d)). Only non-synonymous variants that imply an amino acid change were considered. The putative targets of metformin ranked highly: the gene PRKAB1 which forms part of the AMPK complex, SIRT1 and GPD2. The language model gave higher scores to small molecule tractable targets (CI50% tractability line labelled 502 in FIG. 5(d)). However, there was a depletion of p-values from the GWAS meta-analysis (C199% p-value line labelled 501 in FIG. 5(d)), and none of the putative targets had significant non-synonymous variants. Five variants in four genes (Table 4) met several criteria: non-synonymous, p-value lower than 10e−9, two studies with the same sign in the beta coefficient, and high likelihood (>0.3) to be the target of metformin. The variant rs8179206 with the highest rank was the Glu77Gly in the gene GCKR. This variant has been linked to triglyceride excess in the blood and obesity. However, no publications link the genetic variants rs7273698 and rs7273698 in the gene CD40, rs61737461 in AEBP1 and rs147387411 ACLY with metformin (FIG. 5(d)). ATP citrate lyase (ACLY) catalyses citrate and coenzyme A into acetyl-CoenzymeA and oxaloacetate in many tissues with high-fat production, including the liver, adipocytes and pancreatic beta cells. Acetyl-CoA serves lipogenesis and cholesterogenesis pathways. ACLY is a promising therapeutic target for cholesterol reduction and protection against artherosclerosis. The ubiquitination of ACLY and subsequent degradation switches cell metabolism from synthesis to oxidation of fatty acids. This switch correlates with the activation of AMPK by metformin: stimulation of fatty acid oxidation, inhibition of cholesterol and triglyceride synthesis, increased glucose uptake in skeletal muscle and insulin sensitivity. The Arg592Gln (rs147387411) mutation lies close to the coenzyme A binding domain but not directly in it. Acetylation at three lysine residues K540, K546, and K554 by the lysine acetyltransferase 2B (KAT2B) has been demonstrated to increase ACLY stability by blocking its ubiquitylation and promoting de-novo lipid synthesis. ACLY is deacetylated and inhibited by SIRT2 but not SIRT1, the putative target of metformin. Arg592Gln ACLY mutant could be differently acetylated, ubiquitinated, or phosphorylated upon metformin treatment. However, further experimental work will be required to confirm its link with metformin.

The results obtained in FIG. 5 are obtained as follows. The data for the compound-protein interactions was gathered from DrugBank, which consists of more than 1100 thousand high-quality, curated, binary compound-protein interactions for human proteins. This dataset contains positive examples. Negative examples were created with combinations of the 400 proteins with most compounds and the 400 least specific compounds. Negative examples could not intersect with the positive set. This sampling technique is more realistic and may lead to more false positives.

Multiple machine learning classifiers were trained to prioritise positive and negative compound-target interactions, including random forest classifiers and logistic regression implemented in Scikit Learn and deep neural networks in TensorFlow. The neural networks have a variable number of layers, dropouts and sizes of their hidden layers. All models have a balanced loss function to account for the imbalance in the dependent variable. All models used are displayed in Table 5 below.

Table 5 shows compound-protein model statistics. Metrics for the testing set only. Results were sorted by the Matthews correlation coefficient (MCC). The best model was the H100-N2-D0.2: a neural network with 100 neurons of hidden size (H), 2 hidden layers (N) to allow for pairwise interactions of the fingerprint of the word embeddings and a 0.2 probability of dropout (D). The immediate best model is the random forest with 100 estimators, Gini entropy for bootstrap selection. All models had a balanced loss function to account for imbalance in the dependent variable. The logistic regression model is better than a simple neural network of 1 hidden layer with a size of 1, as expected.

TABLE 5

| Model | Matthews correlation | Precision | Recall |
|---|---|---|---|
| H100-N2-D0.2 | 0.345 | 0.174 | 0.876 |
| RFC | 0.344 | 0.261 | 0.549 |
| H100-N2-D0.0 | 0.344 | 0.172 | 0.882 |
| H100-N3-D0.0 | 0.343 | 0.172 | 0.872 |
| H50-N3-D0.0 | 0.342 | 0.175 | 0.856 |
| H50-N3-D0.2 | 0.333 | 0.164 | 0.878 |
| H50-N2-D0.0 | 0.325 | 0.159 | 0.873 |
| H50-N2-D0.2 | 0.319 | 0.153 | 0.886 |
| H100-N3-D0.2 | 0.318 | 0.152 | 0.888 |
| H100-N1-D0.2 | 0.308 | 0.149 | 0.862 |
| H100-N1-D0.0 | 0.307 | 0.147 | 0.871 |
| H50-N1-D0.0 | 0.307 | 0.149 | 0.859 |
| H50-N1-D0.2 | 0.307 | 0.149 | 0.856 |
| H10-N3-D0.2 | 0.287 | 0.144 | 0.802 |
| H10-N2-D0.0 | 0.281 | 0.132 | 0.853 |
| H10-N2-D0.2 | 0.273 | 0.131 | 0.83 |
| H10-N1-D0.0 | 0.269 | 0.128 | 0.83 |
| H10-N3-D0.0 | 0.265 | 0.123 | 0.847 |
| H10-N1-D0.2 | 0.265 | 0.129 | 0.805 |
| H1-N1-D0.2 | 0.257 | 0.13 | 0.764 |
| Logistic Regression | 0.249 | 0.117 | 0.821 |
| H1-N1-D0.0 | 0.244 | 0.121 | 0.77 |
| H1-N2-D0.0 | 0.177 | 0.086 | 0.756 |
| H1-N2-D0.2 | 0.168 | 0.079 | 0.795 |
| H1-N3-D0.2 | 0 | 0 | 0 |
| H1-N3-D0.0 | 0 | 0 | 0 |

The latest release of the InterPro database was used for protein-motif links. InterPro classifies protein amino acid sequences into families and predicts the presence of functionally essential domains and sites. For the small molecule tractability, the implementation of ‘A Multilayer Network Approach for Guiding Drug Repositioning in Neglected Diseases’, Berenstein et al., PLoS Negl. Trop. Dis. 10, e0004300 (2016) was followed, where each protein target had a tractability score. Each protein had the maximum tractability score from the p-value of a Fisher's exact test for their constituent InterPro motifs. InterPro motifs were associated with compounds through a motif-protein-compound graph. This approach gives non-trivial scores to proteins with druggable motifs if another protein with the same motif has been drugged.

In the above examples, Examples 1 to 4, language models were used to learn from 34 million publications and prioritise the next most likely hypotheses that have not been published yet in different settings: gene-disease associations, target-disease into clinical trials, protein-protein interactions, and unexplored but likely mechanisms of action of a drug. The examples show that biomedical knowledge in the published literature can be encoded as word embeddings or vector representations of words and that without any direct insertion of a priori biomedical knowledge, these embeddings can capture complex biomedical concepts such as drug target classes, tractability, involvement in disease phenotypes, and protein-protein interactions. Furthermore, language models ranked novel hypotheses several years before their publication in top tier journals. This prioritisation suggests that knowledge regarding future discoveries is, to a certain extent, embedded in past publications. To the extent of the literature review performed, this is the first attempt to use a language model to prioritise drug discovery hypotheses before they are published.

In relation to target prioritisation, the vector representations of genes contain information about small molecule and antibody druggability, disease association, therapeutic target family, pathways and clinical precedence that can be generalised to unseen genes. These embeddings contained more information than manually engineered features by biology experts. Around 50% of the top ranked genes ended up being published as associated with the disease within 10 years. Furthermore, some relevant gene-disease associations validated could have been prioritised one to several years before their publication. The described method outperformed the Paliwal et al. method at prioritising clinical trial targets for 200 diseases in the test set.

In relation to protein interactions, the described method outperformed other computational methods like L3. Language models can prioritise protein-protein interactions ahead of time and could complement experiments to complete the human interactome.

In relation to drug target interactions, language models were able to learn known target-drug relationships for specific compounds. Importantly, the putative targets of metformin that were prioritised by language models, and the genome-wide ranking generated, correlated with independent small molecule tractability scores. Arg592Gln ACLY mutant could be differently acetylated, ubiquitinated, or phosphorylated upon metformin treatment.

Latent knowledge regarding future discoveries is to some extent embedded in past publications. Furthermore, language models offer a possibility of prioritising not-yet-proven hypotheses from an increasing body of scientific biomedical literature. This literature-wide association method prioritises therapeutic drug hypotheses that can be later validated with real-world data. Scientific progress and hypothesis generation for target analysis relies on efficiently assimilating the existing knowledge and then choosing the most promising way forward and minimising re-invention. As the amount of biomedical literature keeps rising, this is becoming increasingly difficult, if not impossible, for an individual scientist. This method assists in making the vast amount of information found in scientific literature accessible to individual target analysts in ways that enable a new paradigm of machine-assisted novel target identification breakthroughs. The described method is a scalable system that allows prioritisation of under-explored targets, accelerating early-stage drug target selection agnostic of the disease of interest.

1. A method for drug target selection, the method comprising computer-implemented steps of:
ingesting biomedical publication data from at least one biomedical publication data source that includes text-based biomedical publication documents;
searching the biomedical publication data to identify occurrences of terms indicating a plurality of genes and occurrences of terms indicating at least one disease;
defining a vocabulary comprising a plurality of terms from the biomedical publication data, the plurality of terms comprising the terms indicating the plurality of genes and the terms indicating the at least one disease;
training a defined language model using the vocabulary to obtain vector representations of the plurality of terms; and
for the at least one disease:
determining likelihood scores for the plurality of genes based on the vector representations, the likelihood scores indicating likelihoods of the plurality of genes co-occurring with the at least one disease in the biomedical publication data;
generating a ranking of the plurality of genes according to the likelihood scores; and
selecting at least one gene from the plurality of genes as a drug target for the at least one disease based on the ranking.

2. (canceled)

3. (canceled)

4. The method of claim 1, further comprising computer implemented steps of:
searching the biomedical publication data to identify occurrences of a set of terms indicating a gene of the plurality of genes; and
converting the occurrences of the set of terms indicating the gene to a standard gene term.

5. The method of claim 1, further comprising computer implemented steps of:
searching the biomedical publication data to identify occurrences of a set of terms indicating a disease; and
converting the occurrences of the set of terms indicating the disease to a standard disease term.

6. (canceled)

7. The method of claim 1, further comprising computer-implemented steps of:
searching the biomedical publication data by identifying terms occurring at least a threshold number of times in the biomedical publication data; and
defining the vocabulary from the terms occurring at least the threshold number of times.

8. The method according of claim 4, further comprising computer-implemented steps of:
searching the biomedical publication data by identifying terms comprising unigrams; and
generating phrases of n-grams, where n is greater than or equal to two, based on the unigrams.

9. (canceled)

10. (canceled)

11. The method of claim 1, further comprising computer-implemented steps of generating the vector representations utilizing weights of neurons of a neural network.

12. (canceled)

13. (canceled)

14. The method of claim 1, wherein training the defined language model comprises:
extracting contextual data for the plurality of terms from the biomedical publication data; and
generating, utilizing the defined language model, a prediction of the plurality of terms from the contextual data.

15. (canceled)

16. (canceled)

17. (canceled)

18. (canceled)

19. The method of claim 1, wherein, for the at least one disease, determining the likelihood scores for the plurality of genes comprises determining a distance metric between a vector representation of the at least one disease and the vector representations of the plurality of genes.

20. (canceled)

21. (canceled)

22. (canceled)

23. (canceled)

24. (canceled)

25. The method of claim 1, further comprising steps of: designing a drug discovery project for developing a therapeutic drug for the at least one disease, the drug discovery project being based on the at least one gene being the drug target for the drug discovery project.

26. The method of claim 25, further comprising steps of: undertaking the drug discovery project using the at least one gene as the drug target.

27. The method of claim 26, wherein undertaking the drug discovery project comprises selecting and testing compounds against the at least one gene for therapeutic effects against the at least one disease.

28. The method of claim 27, wherein testing the compounds comprises synthesizing a compound of the compounds and determining one or more properties of the compound.

29. (canceled)

30. (canceled)

31. (canceled)

32. (canceled)

33. The method of claim 8, further comprising computer-implemented steps of defining the vocabulary from the phrases of n-grams based on determining that the phrases of n-grams occur a threshold number of times.

34. The method of claim 8, further comprising computer-implemented steps of defining the vocabulary from the phrases of n-grams based on determining that the phrases of n-grams satisfy a threshold mutual information score.

35. The method of claim 1, wherein training the defined language model comprises generating, utilizing the defined language model, a plurality of probability distributions of contextual data from the plurality of terms.

36. A method for drug target selection, the method comprising computer-implemented steps of:
ingesting biomedical publication data from at least one biomedical publication data source that includes text-based biomedical publication documents;
searching the biomedical publication data to identify occurrences of terms indicating a plurality of genes and occurrences of terms indicating at least one disease;
defining a vocabulary comprising a plurality of terms from the biomedical publication data, the plurality of terms comprising the terms indicating the plurality of genes and the terms indicating the at least one disease;
training a defined language model using the vocabulary;
generating, utilizing the defined language model, vector representations of the plurality of terms; and
for the at least one disease:
determining likelihood scores for the plurality of genes based on the vector representations, the likelihood scores indicating likelihoods of the plurality of genes co-occurring with the at least one disease in the biomedical publication data; and
selecting at least one gene from the plurality of genes as a drug target for the at least one disease based on the likelihood scores.

37. The method of claim 36, wherein training the defined language model comprises:
extracting contextual data for the plurality of terms from the biomedical publication data; and
generating, utilizing the defined language model, a prediction of the plurality of terms from the contextual data.

38. The method of claim 36, wherein training the defined language model comprises generating, utilizing the defined language model, a plurality of probability distributions of contextual data from the plurality of terms.
